(19) United States
(12) Patent Application Publication
(10) Pub. No.: US 2017/0267538 A1
(43) Pub. Date: Sep. 21, 2017

(54) INTEGRATED PROCESS FOR CAPTURING CARBON DIOXIDE

(71) Applicant: Ethan Novek, Greenwich, CT (US)

(72) Inventor: Ethan Novek, Greenwich, CT (US)

(21) Appl. No.: 15/424,218

(22) Filed: Feb. 3, 2017

Related U.S. Application Data
(63) Continuation-in-part of application No. 14/826,771, filed on Aug. 14, 2015, now Pat. No. 9,624,111.
Application No. 14/826,771 is a continuation-in-part of application No. PCT/US2015/064669, filed on Dec. 9, 2015.
Application No. PCT/US2015/064669 is a continuation of application No. 14/826,771, filed on Aug. 14, 2015, now Pat. No. 9,624,111.
(60) Provisional application No. 62/290,519, filed on Feb. 3, 2016.
(60) Provisional application No. 62/375,471, filed on Aug. 16, 2016.
(60) Provisional application No. 62/106,822, filed on Jan. 23, 2015.
(60) Provisional application No. 62/090,272, filed on Dec. 10, 2014.
(60) Provisional application No. 62/159,481, filed on May 11, 2015.
(60) Provisional application No. 62/309,666, filed on Mar. 17, 2016.
(60) Provisional application No. 62/325,620, filed on Apr. 21, 2016.
(60) Provisional application No. 62/363,445, filed on Jul. 18, 2016.

(51) Int. Cl. C01C 1/26; C07C 273/10; C07C 269/06; C07C 269/00; H02K 7/18; C07C 273/04

(52) U.S. Cl. CPC C01C 1/26; C01B 31/20; Y02P 20/125; H02K 7/1823; C07C 273/10; C07C 273/04; C07C 269/06; C07C 269/00

(57) ABSTRACT

The invention pertains to an integrated process for capturing CO₂. The process involves desorbing gaseous CO₂ from a CO₂ containing aqueous solution comprising carbonate, bicarbonate, sesquicarbonate, carbamate, or a mixture thereof. The desorbing of gaseous CO₂ is conducted in the presence of a suitable water soluble substance. If desired, the process may also at least partially recover the soluble substance using a membrane, distillation, or another technique.

### CROSS REFERENCE TO RELATED APPLICATIONS

The instant application claims priority to U.S. Ser. No. 62/290,519 filed Feb. 3, 2016. This application is related to U.S. Ser. No. 14/826,771 filed Aug. 14, 2015 which claims priority to U.S. Ser. Nos. 62/106,822 filed Jan. 23, 2015 and 62/090,272 filed Dec. 10, 2014 and 62/159,481 filed May 11, 2015. This application is also related to PCT/US2015/064669 filed Dec. 9, 2015 claiming priority to U.S. Ser. No. 14/826,771 filed Aug. 14, 2015 which claims priority to U.S. Ser. Nos. 62/106,822 filed Jan. 23, 2015 and 62/090,272 filed Dec. 10, 2014 and 62/159,481 filed May 11, 2015. All of the aforementioned applications are incorporated herein by reference.

### BACKGROUND AND SUMMARY OF INVENTION

Among human activities, CO₂ emissions from electricity generation and industry make up 65% of global greenhouse gas emissions. Considering the world's growing energy demand and continued dependence on fossil fuels, there is an unprecedented need to develop technologies to significantly reduce CO₂ emissions.

One promising means of reducing CO₂ emissions is post-combustion CO₂ capture and utilization (CCU), which transforms low concentrations of CO₂ in emissions into high purity CO₂ for utilization or disposal. However, implementation of these technologies, such as the chilled ammonia and monoethanolamine (MEA) carbon capture processes, has been limited to pilot plants due to enormous operating costs. The most effective current processes require high temperature heat, generally supplied by steam diverted from power generation, increasing electricity costs by over 70% in some cases. High temperature heat constitutes >80% of the energy consumption in current carbon capture processes and is the costliest component of CO₂ capture. A significantly lower operating and capital cost CO₂ capture system is necessary to make CCU an effective means of reducing CO₂ emissions.

Pure CO₂ is a valuable product with 80 Mt per year commercial market. Due to the cost prohibitive nature of current CO₂ capture systems, over 80 percent of the demand for pure CO₂ is supplied by the unsustainable drilling of CO₂ source fields, which contain CO₂ that has been sequestered for millions of years. An effective system that captures CO₂ from flue gas below market prices would at least partially displace the pure CO₂ production from these unsustainable and counterproductive sources.

Advantageously, the present invention pertains to a new highly efficient, low energy, and low cost system and methods to capture CO₂ from one or more CO₂ containing gas mixtures. CO₂ is absorbed from a gas mix containing CO₂, such as flue gas, by a CO₂-lean solution comprising one or more CO₂ absorbents. Then a soluble substance is added to the resulting CO₂ rich solution, eliciting the desorption of gaseous carbon dioxide. The resultant solution following the aforementioned CO₂ desorption is separated into the soluble substance and the CO₂-lean absorption solution. The CO₂-lean absorption solution is transferred to the absorption step and the soluble substance is transferred to the CO₂ desorption step, making the process regenerable. The soluble substance and the CO₂-lean absorption solution recovery is characterized by one or more or a combination of the following:
(a) Membrane—Based Separation comprising one or a combination of the following:
   a. Nanofiltration
   b. Organic Solvent Nanofiltration
   c. Reverse Osmosis
   d. Forward Osmosis
   e. Ultrafiltration
   f. Microfiltration
(b) Distillation comprising one or a combination of the following:
   a. Batch distillation
   b. Continuous distillation
   c. Simple distillation
   d. Fractional distillation
   e. Steam distillation
   f. Azeotropic distillation
   g. Multi-effect distillation
   h. Multi-stage flash distillation
   i. Flash distillation
   j. Mechanical vapor compression distillation
   k. Membrane distillation
   l. Vacuum distillation
   m. Short path distillation
   n. Zone distillation
   o. Air sensitive distillation
(c) Switchable solvent—one or a combination of the following:
   a. Thermally switchable
   b. CO₂-switchable
   c. Switchable solvents responsive to other changes to system conditions.

In one embodiment the invention pertains to an integrated process for capturing CO₂. The process comprises desorbing gaseous CO₂ from a CO₂ containing aqueous solution comprising carbonate, bicarbonate, sesquicarbonate, carbamate, or a mixture thereof. The desorbing of gaseous CO₂ is conducted in the presence of a suitable water soluble substance.

In another embodiment the invention pertains to an integrated process for capturing CO₂. The process comprises capturing CO₂ to form a CO₂ containing solution comprising carbonate, bicarbonate, sesquicarbonate, carbamate, or a mixture thereof. Gaseous CO₂ is desorbed from the CO₂ containing solution comprising carbonate, bicarbonate, sesquicarbonate, carbamate, or a mixture thereof. The desorbing of gaseous CO₂ is conducted in the presence of a suitable soluble substance. The soluble substance is at least partially recovered by employing (1) a membrane with a molecular weight cutoff of greater than about 80 daltons or (2) distillation or (3) a combination thereof.

The soluble substance may comprise water, organic solvent, siloxanes, ionic liquids, water soluble polymer, soluble polymer, glycol, polyethylene glycol, polypropylene glycol, ethers, glycol ethers, glycol ether esters, triglyme, polyethylene glycols of multiple geometries, including, branched polyethylene glycols, star polyethylene glycols, comb polyethylene glycols, methoxypolyethylene glycol, polyvinyl alcohol, polyvinylpyrrolidone, polyacrylic Acid, diol polymers, 1,2 propanediol, 1,2 ethanediol, 1,3 propanediol, cellulose ethers, methylcellulose, cellosize, carboxymethylcellulose, hydroxyethylcellulose, sugar alcohol, sugars, alcohols, ketones, aldehydes, esters, organosilicon compounds, halogenated solvents, non-volatile solvents, a substance with a vapor pressure less than 0.01 atm at 20° C., soluble substances with a molecular weight greater than 80 daltons, volatile organic solvents, soluble substances with a molecular weight less than 600 daltons, soluble substances with a molecular weight less than 200 daltons, dimethoxymethane, acetone, acetaldehyde, methanol, dimethyl ether, THF, ethanol, isopropanol, propanal, methyl formate, azeotropes, alcohols, ketones, aldehydes, esters, organosilicon compounds, halogenated solvents, a substance with a vapor pressure greater than than 0.01 atm at 20° C., or a mixture thereof.

### BRIEF DESCRIPTION OF THE DRAWINGS

FIG. 1 illustrates an embodiment of a CO₂ capture system with CO₂ desorption in the presence of a soluble substance and membrane-based recovery.

FIG. 2 illustrates an embodiment of a CO₂ capture system with CO₂ desorption in the presence of a volatile soluble substance and distillation recovery.

FIG. 3 illustrates an embodiment of a CO₂ capture system with CO₂ desorption in the presence of a CO₂ switchable solvent and thermal CO₂ switching recovery.

FIG. 4 illustrates an embodiment of a CO₂ capture system with CO₂ desorption in the presence of a CO₂ switchable solvent and air-contacting CO₂ switching recovery.

FIG. 5 illustrates an embodiment of a CO₂ capture system with CO₂ desorption in the presence of a thermally switchable solvent and thermal switching recovery.

FIG. 6 illustrates an embodiment of a CO₂ capture system with CO₂ desorption in the presence of a soluble substance and hybrid ‘salting-out’ and membrane recovery.

FIG. 7 illustrates an embodiment of a CO₂ capture system with CO₂ desorption in the presence of an ultra-low boiling point water soluble substance and mechanical vapor compression distillation.

FIG. 8 illustrates an embodiment of a CO₂ capture system with CO₂ desorption in the presence of an ultra-low boiling point water soluble substance and mechanical vapor compression distillation wherein heat is exchanged between the distillation and absorption stages, chilling the absorption stage.

FIG. 9 illustrates an embodiment of a CO₂ capture system with CO₂ desorption in the presence of a soluble substance and nanofiltration membrane recovery, wherein the nanofiltration stage is heated.

FIG. 10 shows rate of CO₂ desorption in specific experiments.

FIG. 11 shows CO₂ desorbed in specific experiments.

FIG. 12 shows CO₂ generated at different ammonium bicarbonate solution concentrations.

FIG. 13 shows a plateau in CO₂ generations at high ammonium carbonate and solvent concentrations.

FIG. 14A shows CO₂ release as a function of final solvent mole fraction for 2M ammonium bicarbonate.

FIG. 14B shows CO₂ release as a function of final solvent mole fraction for 1M ammonium bicarbonate.

FIG. 15A shows reboiler temperature requirement in a chilled ammonia process.

FIG. 15B shows reboiler temperature requirement for acetone and DMM.

### DETAILED DESCRIPTION OF THE INVENTION

The instant invention generally pertains to an integrated process for capturing CO₂. The process comprises desorbing gaseous CO₂ from a CO₂ containing solution comprising carbonate, bicarbonate, sesquicarbonate, carbamate, or a mixture thereof. The desorbing of gaseous CO₂ is usually conducted in the presence of a suitable soluble substance.

The CO₂ containing solution may be formed in any convenient manner. Generally, any solution capable of dissolving CO₂ in desirable amounts may be employed. The components and amounts of such solutions may vary depending upon factors such as, for example, the amount of CO₂ to be dissolved, the source and state of CO₂ and any impurities therewith, the specific desorbing steps, any subsequent processing steps, and other factors.

Typically, the CO₂ containing solution may be derived from or comprise a CO₂ absorbent that is capable of capturing CO₂ from the desired source at the desired parameters. Such absorbents may vary widely depending upon the source and desired characteristics of the CO₂ containing solution to be formed. Typically the CO₂ absorbent may comprise, for example, water, ammonia, ammonium, amine, azine, amino ethyl ethanol amine, 2-amino-2-methylpropan-1-ol (AMP), MDEA, MEA, primary amine, secondary amine, tertiary amine, low molecular weight primary or secondary amine, metal-amine complex, metal-ammonia complex, metal-ammonium complex, sterically hindered amine, imines, azines, piperazine, alkali metal, lithium, sodium, potassium, rubidium, caesium, alkaline earth metal, calcium, magnesium, ionic liquid, thermally switchable compounds, CO₂ switchable compounds, enzymes, metal-organic frameworks, quaternary ammonium, quaternary ammonium cations, quaternary ammonium cations embedded in polymer, or mixtures thereof.

The amounts of CO₂ to be captured from the source will vary. Typically, it is desired to capture at least about any of the following percentages (%) from the total CO₂ in the source: 40, or 50, or 60, or 70, or 80, or 90, or substantially 100.

The CO₂ may be captured from any convenient source using any convenient manner. If desired, the CO₂ source may be treated, e.g., scrubbed, before being subjected to the absorbent and/or forming the CO₂ containing solution. Such treating methods may be particularly advantageous if the source has impurities that may deleteriously affect subsequent processing steps, e.g., recovery steps employing a membrane or distillation. Such impurities include, but are not limited to, NOx, SOx, oils, particulate matter, heavy metals, and heavy compounds, etc. Conventional treating methods may be employed for this purpose.

If desired, the CO₂ source may be left untreated or only partially treated before being subjected to the absorbent and/or forming the CO₂ containing solution. Such an instance may be particularly advantageous if the source does not have impurities or has impurities which are benign or have ameliorable affects. Such an example may include a CO₂ source containing NOx or SOx, which may be subjected to a CO₂ absorbent comprising of aqueous ammonia. The NOx or SOx may react with said ammonia, forming salable products, such as ammonium nitrate, ammonium sulfate, ammonium sulfite, ammonium bisulfite, ammonium metabisulfite or ammonium nitrite. Said salable byproducts may be removed by any convenient manner, including, but not limited to, ion exchange, ion exchange membrane, electrodialysis, or removal or replacement of the absorbent and/or CO₂ containing solution.

Convenient sources from which to capture CO₂ for the CO₂ containing solution include sources selected from the group consisting of flue gas; combustion emissions; manufacturing emissions; refining emissions or a combination thereof. Such sources may include, for example, from combustion of one or more hydrocarbons; emissions from the combustion of natural gas, coal, oil, petcoke, gasoline, diesel, biofuel, or municipal waste; emissions from waste water treatment gases, or landfill gases, from air, from metal production/refining, from the production of Iron, Steel, Aluminum or Zinc, from cement production, from quicklime production, from Glass production, oil and gas refineries, steam reforming, hydrogen production, HVAC, refrigeration, transportation vehicles (ships, boats, cars, buses, trains, trucks, airplanes), natural gas, biogas, alcohol fermentation, volcanic activity, decomposing leaves/biomass, septic tank, respiration, manufacturing facilities, fertilizer production, geothermal wells, and combinations thereof.

Once the CO₂ is captured the CO₂ containing solution may typically comprise carbonate, bicarbonate, sesquicarbonate, carbamate, or a mixture thereof. Of course, the solution may also comprise suitable cations such as ammonium and other species such as described above that may remain from any CO₂ absorbent. Generally, the CO₂ containing solution may be aqueous, but, of course, it may take other forms as well depending upon the embodiment employed.

The desorbing of gaseous CO₂ may be conducted in any convenient manner. Such manner will vary depending upon the specific amount, composition, and nature of the CO₂ containing solution. Typically, the desorbing is conducted in the presence of a suitable soluble substance, for example, water soluble substance. Useful substances and potentially useful concentrations vary depending upon the reactants, amounts, and desired outcomes. The specific manner of combining the suitable soluble substance and CO₂ containing solution is not particularly critical in most instances. That is, the suitable soluble substance may be added to the CO₂ containing solution, the CO₂ containing solution may be added to the suitable soluble substance, or one or the other could even be formed in situ or combined in some other manner.

The amounts of CO₂ to be desorbed will vary. Typically, it is desired to desorb at least about any of the following percentages (%) from the total CO₂ in the source: 40, or 50, or 60, or 70, or 80, or 90, or substantially 100%.

The soluble substance employed may vary depending upon, for example, whether it is to be at least partially recovered, and, if so, in what manner. By “at least partially recovered” it is meant from at least about 50%, or at least about 60%, or at least about 70%, or at least about 80%, or at least about 90%, or at least about 95%, or at least about 99% up to nearly 100% of the soluble solvent is recovered for re-use in the process or something else.

The manner of at least partially recovering the soluble substance is not particularly critical and will vary depending upon such factors as the specific composition, the desired outcome, and equipment available. For example, the separation mechanism used for at least partially recovering the soluble substance may include one or more or a combination of the following: membrane, reverse osmosis, hot reverse osmosis, nanofiltration, organic solvent nanofiltration, hot nanofiltration, ultrafiltration, hot ultrafiltration, microfiltration, filtration, distillation, membrane distillation, multi-effect distillation, mechanical vapor compression distillation, binary distillation, azeotrope distillation, hybrid separation devices, flash distillation, multistage flash distillation, extractive distillation, switchable solvent, ‘salting-out,’ or centrifuge, or combinations thereof.

In one embodiment the soluble substance may be at least partially recovered by employing a membrane that is, for example, capable of at least partially rejecting said soluble substance while allowing substantial passage of CO₂ containing aqueous solution or vice versa. “CO₂ containing solution” or “CO₂ containing aqueous solution” simply refers to the subsequently obtained solution after desorbing of CO₂. Thus, CO₂ containing aqueous solution or CO₂ containing solution may have various amounts of CO₂ or even no CO₂ depending upon the amount of CO₂ desorbed in the desorbing step. This subsequently obtained solution typically comprises the solution components less any CO₂ desorbed while any soluble substance is at least partially recovered by virtue of being rejected by the membrane. In such embodiments the soluble substance may comprise, for example, water, organic solvent, water soluble polymer, soluble polymer, glycol, polyethylene glycol, polypropylene glycol, ethers, glycol ethers, glycol ether esters, triglyme, polyethylene glycols of multiple geometries, including, branched polyethylene glycols, star polyethylene glycols, comb polyethylene glycols, methoxypolyethylene glycol, polyvinyl alcohol, polyvinylpyrrolidone, polyacrylic Acid, diol polymers, 1,2 propanediol, 1,2 ethanediol, 1,3 propanediol, cellulose ethers, methylcellulose, cellosize, carboxymethylcellulose, hydroxyethylcellulose, sugar alcohol, sugars, alcohols, ketones, aldehydes, esters, organosilicon compounds, halogenated solvents, non-volatile solvents, a substance with a vapor pressure less than 0.01 atm at 20° C., soluble substances with a molecular weight greater than 80 daltons, or mixtures thereof.

Useful membranes for at least partial recovery may include, for example, any membrane capable of at least partially rejecting said soluble substance while allowing substantial passage of CO₂ containing aqueous solution or vice versa. Such membranes may comprise a membrane selected from the group consisting of Reverse Osmosis, Nanofiltration, Organic Solvent Nanofiltration, Ultrafiltration, Microfiltration, and Filtration membranes. In some embodiments the membrane may have a molecular weight cutoff of greater than about 80 daltons. That is, the membrane allows passage of a substantial or majority amount of components with a molecular weight of less than about 80 daltons while rejecting a substantial or majority amount of components with a molecular weight of greater than about 80 daltons up to about 600 daltons. In the art, another definition of molecular weight cut-off may refer to the lowest molecular weight solute (in daltons) in which 90% of the solute is retained by the membrane, or the molecular weight of the molecule that is 90% retained by the membrane. Membranes with a molecular weight cutoff of less than 1,000 daltons, or less than 10,000 daltons, or less than 50,000 daltons, or less than 100,000 daltons, or less than 200,000 daltons, or less than 500,000 daltons, or less than 1,000,000 daltons may also be useful depending upon the circumstances and components employed.

The membrane may be comprised of any useful material and such useful material may vary depending upon the components to be separated, their molecular weight, viscosity, and/or other properties. Useful membranes may include, for example, membranes comprised of a material selected from a thin film composite; a polyamide; a cellulose acetate; a ceramic membrane; other materials and combinations thereof.

Generally, it may be preferred to select membranes, substances, and conditions such that any at least partial recovery step(s) involving one or more membranes may be conducted at a temperature of less than or equal to about 50, or less than or equal to 40, or less than or equal to about 35, or less than or equal to about 30° C. In other specific embodiments the at least partial recovery step(s) temperature may be at a temperature of from about 18° C. to about 32° C. Similarly, the pressure employed during any at least partial recovery may be any convenient pressure, e.g., elevated, reduced, or substantially atmospheric. For example, the step(s) may be conducted at a pressure of from about 0.75 to about 1.25 atmospheres. In another embodiment the at least partial recovery conditions employing one or more membranes are substantially room temperature and pressure.

In another embodiment the at least partially recovering said soluble substance may be accomplished by distillation or some equivalent thereof. In such embodiments the soluble substance may comprise, for example, one or more or a combination of the following: volatile organic solvents, soluble substances with a molecular weight less than 600 daltons, soluble substances with a molecular weight less than 200 daltons, dimethoxymethane, acetone, acetaldehyde, methanol, dimethyl ether, THF, ethanol, isopropanol, propanal, methyl formate, azeotropes, alcohols, ketones, aldehydes, esters, organosilicon compounds, halogenated solvents, a substance with a vapor pressure greater than 0.01 atm at 20° C., or a mixture thereof.

The integrated process wherein CO₂ volatilizes may occur in the presence of a low CO₂ partial pressure gas, in the presence of air, with the application of heat, or a combination thereof.

If distillation is to be employed then often the distillation of the substance to be at least partially received depends upon the components and may occur at a temperature of less than about 110° C., or less than about 100° C., or less than about 90° C., or less than about 80° C., or less than about 70° C., or less than about 60° C., or less than about 50° C., or less than about 40° C., or less than about 30° C.

In some embodiments the soluble substance may comprise a thermally switchable substance, a CO₂ switchable substance, or a non-ionic carbon containing compound. A switchable substance is one which substantially separates from other materials depending upon, for example, a property or other ingredients of a combined composition. That is, a thermally switchable substance may precipitate from a given solution when subjected to temperatures above or below a certain threshold, e.g., cloud point. Useful thermally switchable substances may include, for example, those that substantially precipitate, separate, or have a cloud point at or above 30, or 40, or 50, or 60, or 70, or 80, or 90, or 100, or 110° C.

The integrated process may be conducted with a CO₂-switchable substance as the soluble substance. Such substances may precipitate or separate depending upon the amount of CO₂ dissolved in the solution. That is, a CO₂-switchable substance may be soluble in solutions such as aqueous solutions when sufficient CO₂ is dissolved but separate and become insoluble upon release of sufficient gaseous CO₂. For example, the switchable solvent may be hydrophobic upon volatilization of substantial amounts, e.g., a majority, of dissolved CO₂.

The concentration of the soluble substance(s) and any CO₂ absorbent employed in the integrated process may vary depending upon the substance, other substances, and desired results. Typically, each may have a concentration of from about 1M to about 18M. That is, the concentration of each may be independent or dependent of the other and be, for example, greater or less than 1M, or less than 2M, or less than 3M, or less than 4M, or less than 5M, or less than 6M or less than 10M up to as high as 18M.

The specific desorbing conditions may vary depending upon the amount of CO₂ present, the soluble substance employed and its concentration, the absorbent precursor or residual, if any, and its concentration, the presence and type of any impurities, the desired partial recovery steps, if any, and other factors. Generally, it may be preferred to select substances and conditions such that the desorbing step may be conducted at a temperature of less than or equal to about 50, or less than or equal to 40, or less than or equal to about 35, or less than or equal to about 30° C. In other embodiments the desorbing temperature may be at a temperature of from about 18° C. to about 32° C. Similarly, the pressure employed may be any convenient pressure. For example, the CO₂ may be desorbed at a pressure of from about 0.75 to about 1.25 atmospheres. In another embodiment the desorbing conditions are substantially room temperature and pressure.

The integrated process of the present invention may involve further comprising making additional useful compounds from the solution, CO₂, or both. That is, further processing steps may comprise producing ammonium carbamate, urea, or a derivative thereof.

### General Description of Specific Embodiments

1) Absorption (stage 1): Flue gas enters one or more absorption columns and carbon dioxide is absorbed in a CO₂-lean aqueous CO₂ absorbent-carbon dioxide solution, forming a CO₂-rich aqueous solution. Any remaining inert gases from the flue gas, such as N₂, O₂, Ar, low concentrations of CO₂, may be released from the absorption column and may undergo further treatment. The CO₂-rich solution created in the absorption stage can be transferred to the CO₂ desorption stage (stage 2).

2) CO₂ Desorption (stage 2): A soluble substance, such as an organic solvent or water soluble polymer as described above, is added and mixed with the CO₂-rich solution under, for example, room temperature and pressure conditions. CO₂(g) is desorbed from the solution and may undergo compression or other treatment prior to utilization or conversion. After CO₂ desorption, the CO₂-lean solution comprising the soluble substance can be transferred to soluble substance and CO₂ absorption solution recovery stage (stage 3).

3) Soluble Substance and CO₂ Absorbing Solution Recovery (stage 3): The CO₂-lean solution containing the soluble substance is separated into the CO₂-lean absorption solution and the soluble substance using one or more separation mechanisms or devices. The CO₂-lean absorption solution can be circulated to stage 1 and the soluble substance can be circulated to stage 2. Stage 3 allows the integrated process to be as regenerable as desired.

### Carbon Dioxide Absorption:

Carbon dioxide absorption with examples employing aqueous ammonia or amine species solutions involve absorbing CO₂ from CO₂(g) containing gas streams in a lean solution to create a rich solution. The lean solution may have a CO₂ loading comprising between 0.2-0.67 and the rich solution may have a CO₂ loading comprising between 0.45-1. The molar ratio may differ depending on the embodiment and CO₂ absorbent or absorbents employed. Greater CO₂ loading in the CO₂ rich solution may be achieved by, including, but not limited to, changing the temperature, increasing pressure, increasing CO₂ partial-pressure, increasing contact time, increasing residence time, increasing packing surface area, and/or the addition of a catalyst that accelerates CO₂ absorption.

The absorption tower may be chilled to reduce absorbent volatilization, such as ‘ammonia-slip’ or the volatilization of other components of the absorption media. Absorbent volatilization may also be reduced by operating the absorption solutions at a greater CO₂ loading, although this may result in lower absorption rates and CO₂ absorption capacity. CO₂ loading may be optimized to maximize reaction kinetics and solution capacity. The absorption column may absorb less than or equal to any of the following: 5%, or 10% or 20%, or 30%, or 40%, or 50%, or 60%, or 70%, or 80% or 90%, or 99%, or 99.9%, or 100% of the CO₂ from the CO₂ containing gas stream.

The absorption stage may include any absorption setup known in the art and may be composed of one or more absorption columns or vessels or other devices. The absorption column may include, but is not limited to, continuous absorption, continuous stirred absorption, batch column, packed column, plate column, hybrid absorption processes and other absorption processes known in the art. The absorption column or absorption solution may be chilled, wherein cooling may be conducted via any means including, but not limited to, ambient source, water bodies, cooling tower, industrial evaporative chiller and other chilling or cooling processes known in the art. It may be desirable for the CO₂ concentration in the CO₂ lean solution to be less than the CO₂ concentration in the CO₂ rich solution. A CO₂(g) containing gas stream, including but not limited to flue gas, synthesis gas, steam-reforming gas, methane reforming gas, hydrogen production gases, air, concentrated, membrane concentrated gas stream, membrane concentrated flue gas, upstaged air (as would be created from the moisture swing CO₂ upstaging processes described by Klaus Lackner http://pubs.acs.org/doi/abs/10.1021/es201180v, incorporated herein by reference), biogas, landfill gas, or anaerobic digester gas. The CO₂ containing gas stream may be treated, used as an enthalpy, heat or cold source, or otherwise used prior to the absorption stage.

The remaining gas stream after at least a portion of the CO₂(g) is absorbed, or ‘inert gases’ may undergo further treatment or utilization, including but not limited to, thermal exchange with incoming CO₂ lean solution, water wash to remove trace gases, such as ammonia or organic solvent, removal process for trace gases, additional CO₂ scrubbing method, including, but not limited to, amines, solid sorbent, SELEXOL, UCARSOL, membrane or strong base, separation, purification, or use of constituents, such as hydrogen, carbon monoxide, nitrogen, oxygen and/or argon.

Additionally, the remaining gas stream following the absorption column, such as the ‘inert gases,’ which may contain a lower concentration of CO₂ than the entering CO₂ containing gas stream, may be advantageously used in a CO₂ conversion process that benefits from a relatively lower concentration of CO₂, such as biological processes and certain cement production processes. For example, cement production processes that use CO₂ as a reagent, the oxide or silicate or calcium oxide or calcium silicate or magnesium oxide or magnesium silicate containing reactants may initially require only low CO₂ concentrations due to the highly exothermic nature of the reaction to form carbonates. As a significant portion of the reagents react with the CO₂, such as a conversion yield of greater than any of the following: 0.01%, or 1%, or 5%, 10%, or 20%, or 30%, or 40% or 50%, or 60%, or 70%, or 80%, or 90%, or 95% conversion, the unreacted reagents require an increasingly greater concentration of CO₂. This higher purity CO₂ may be supplied by the integrated CO₂ capture process.

Additionally, the absorption column may absorb a smaller percentage of the CO₂ in the CO₂ containing gas stream, such as less than any of the following: 20%, or 30%, or 40% or 50% or 60%, or 70%, or 80%, or 90%, or 99%. This may further reduce energy requirements, including due to the ability for the CO₂ lean and rich solutions to a higher CO₂ loading. The substance addition CO₂ desorption stage may work more efficiently when the CO₂-rich and CO₂-lean solutions are at a relatively higher CO₂-loading. This may also may reduce capital costs by decreasing the require dimensions of the absorption column.

The CO₂-rich solution may exit the absorption column and may be transferred to Step 2. It may be advantageous to heat exchange this CO₂-rich solution with the CO₂-lean solution entering the absorption column. This may include a countercurrent heat exchange, resulting in a cooler/pre-cooled CO₂ lean stream and a warmer/pre-heated CO₂ rich stream.

Prior to entering the CO₂ absorption column, the CO₂ containing gas stream may, if advantageous, be treated, via methods, including but not limited to, chilling and removal of contaminants, such as hydrogen sulfide, NOₓ, SOₓ, particulates and metals. The gas stream may be further concentrated with a gas membrane CO₂ concentrator or moisture-swing CO₂ concentrator. The entering gas stream may be used as an energy source to supplement energy requirements, including, but not limited to, heating or cooling in the integrated process or components of connecting infrastructure, such as piping. This gas stream may be thermally exchanged by means including, but not limited to, a heat exchanger or direct contacting.

Absorption Solution: The absorption solution includes any aqueous or non-aqueous solution which absorbs CO₂. CO₂ absorbents include, but are not limited to, one or more or a combination of the following: water, ammonia, ammonium amine, primary amine, secondary amine, tertiary amine, methylamine (MEA), methylethanolamine, aminoethylethanolamine, azine, imine, strong base, hydroxide, sodium hydroxide, potassium hydroxide, sodium oxide, potassium oxide, organic solvent, commercial CO₂ capture absorbents, quaternary ammonium compound, Selexol, Rectisol, KS-1, UCARSOL, metal-organic framework, solid adsorbent, high surface area compounds, activated carbon, zeolites, carbon nanotubes, graphene, graphene oxide, amine, amino ethyl ethanol amine, 2-Amino-2-methylpropan-1-ol (AMP), MDEA, MEA, primary amine, secondary amine, or tertiary amine, low molecular weight primary or secondary amine, metal-ammine complex, metal-ammonia complex, metal-ammonium complex, sterically hindered amine, imines, azines, piperazine, amine functionalized polymers, alkali metal, lithium, sodium, potassium, rubidium, caesium, alkaline earth metal, calcium, magnesium, cations, ionic liquid, CO₂ switchable solvents, CO₂ switchable surfactants carbonate, polymer containing amine functional groups, poler containing CO₂ reactive functional groups, enzymes, metal-organic frameworks, glycolamine, diglycolamine, quaternary ammonia or quaternary ammonium cations, or quaternary ammonium cations embedded in polymer, or mixtures thereof. CO₂ may be present in solution as one or more species throughout the integrated process, including, but not limited to, one or more or a combination of the following: bicarbonate, carbonate, carbamate, sesquicarbonate, free CO₂, or dissolved CO₂.

Additionally, the absorption solution may contain a desorption, absorption, or adsorption rate promoter, including, but not limited to, piperazine, diethanolamine, diglycolamine, and diisopropanolamine. Rate promoters may be used to, including, but not limited to, influence one or more of the following: CO₂ absorption, CO₂ desorption, soluble substance regeneration or reaction kinetics.

The CO₂ loading of the CO₂-lean solution may be dependent on the amount of CO₂ desorbed during the substance addition CO₂ desorption and the regeneration stages. Therefore, CO₂ loading of CO₂-lean solution may be adjusted through, including, but not limited to, changing one or more or a combination of the following: residence time, added substance type or types, soluble substance concentration in the mixed CO₂ desorption solution, concentration of the soluble substance in the added substance solution, temperature, application of heating or cooling, CO₂ loading in the CO₂ rich solution, pressure, or CO₂ loading in the in the added substance solution.

Small concentrations of soluble substance may persist or be present in the CO₂ absorption solution. Low concentrations of soluble substances, such as organic solvents, may reduce ammonia slip or other CO₂ absorbent volatilization in the absorption column and reduce energy consumption during regeneration. Additionally, low concentrations of soluble substances, such as organic solvents, may increase CO₂ uptake and inhibit unintended CO₂ volatilization. The maximum said low concentration is dependent on the type of substance and includes, but is not limited to, vol/vol % concentrations of less than any of the following: 0.001%, or 0.1%, or 0.5%, or 1%, or 1.5%, or 2%, or 2.5%, or 3%, or 3.5%, or 4%, or 4.5%, or 5%, or 5.5%, or 6%, or 6.5%, or 7%, or 7.5%, or 8%, or 8.5%, or 9%, or 9.5%, or 10%, or 10.5%, or 11%, or 11.5%, or 12%, or 12.5%, or 13%, or 13.5%, or 14%, or 14.5%, or 15%.

Carbon Dioxide Sources: Any process or resource producing or containing carbon dioxide. Examples of CO₂ sources include, but are not limited to, the following: Power Plant (Natural gas, coal, oil, petcoke, biofuel, municipal waste), Waste Water Treatment, Landfill gas, Air, Metal production/refining (such as Iron, Steel, Aluminum, etc.), Glass production, Oil refineries, HVAC, Transportation vehicles (ships, boats, cars, buses, trains, trucks, airplanes), Natural Gas, Biogas, Alcohol fermentation, Volcanic Activity, Decomposing leaves/biomass, Septic tank, Respiration, Manufacturing facilities, Fertilizer production, or Geothermal processes where CO₂(g) releases from a well or wells.

Non-Aqueous Embodiment: The integrated process may be aqueous or non-aqueous. A non-aqueous process may use a non-aqueous solution media as part of the CO₂ containing solution. Media include, for example, polar organic solvents, including, but not limited to, ethylene carbonate, propylene carbonate, ethylene glycol, propylene glycol, DMSO, water and acetonitrile or inorganic solvents, such as liquid ammonia or liquid amines and mixtures thereof. The non-aqueous system may use a solution media containing of one or more CO₂ absorbents, such as ammonia, ammonium, amines or amine functionalized polymers.

CO₂ Absorbent Concentration: CO₂ absorbents may be at a wide range of concentrations. The absorbent concentration may be as a low as 0.000001 M or as great as pure absorbent. In molarity terms, the concentration of the CO₂ absorbent may be as low as 0.00001M or less than any of the following: 0.01 M, or 0.05M, or 0.1M, or 0.3M, or 0.5M, or 0.8 M, or 1M, or 1.3M, or 1.5M, or 1.8M, or 2M, or 2.3M, or 2.5M, or 2.8M, or 3M, or 3.3M, or 3.5M, or 3.8M, or 4M, or 5M, or 6M, or 7M, or 8M, or 9M, or 10M, or 12M, or 15M, or 18M, or even pure absorbent.

In volume/volume % terms, the CO₂ absorbent concentration range may be as low as 0.0001% to as great as 99.99999%. The concentration of the CO₂ absorbent may be as low as 0.001%, or any of the following: 0.01%, or less than 0.1%, or 0.5%, or 1%, or 1.5% or 2%, or 2.5%, or 3%, or 3.5%, or 4%, or 4.5% or 5%, or 5.5%, or 6%, or 6.5%, or 7%, or 7.5%, or 8%, or 8.5%, or 9%, or 9.5%, or 10%, or 10.5%, or 11%, or 11.5%, or 12%, or 12.5%, or 13%, or 13.5%, or 14%, or 14.5%, or 15%, or 15.5%, or 16%, or 16.5%, or 17%, or 17.5% or 18%, or 18.5%, or 19%, or 19.5%, or 20%, or 20.5%, or 21%, or 21.5%, or 22%, or 22.5%, or 23%, or 23.5% or 24%, or 24.5%, or 25%, or 25.5%, or 26%, or 26.5%, or 27%, or 27.5%, or 28%, or 28.5%, or 29%, or 29.5%, or 30%, or 31%, or 32%, or 33%, or 34%, or 35%, or 36%, or 37%, or 38%, or 39%, or 40%, or 41%, or 42%, or 43%, or 44%, or 45%, or 46%, or 47%, or 48%, or 49%, or 50%, or 51%, or 52%, or 53%, or 54%, or 55%, or 56%, or 57%, or 58%, or 59%, or 60%, or 61%, or 62%, or 63%, or 64%, or 65%, or 66%, or 67%, or 68%, or 69%, or 70%, or 71%, or 72%, or 73%, or 74%, or 75%, or 76%, or 77%, or 78%, or 79%, or 80%, or 81%, or 82%, or 83%, or 84%, or 85%, or 86%, or 87%, or 88%, or 89%, or 90%, or 90.5%, or 91%, or 91.5%, or 92%, or 92.5%, or 93%, or 93.5%, or 94%, or 94.5%, or 95%, or 95.5%, or 96%, or 96.5%, or 97%, or 97.5%, or 98%, or 98.5%, or 99%, or 99.5%, or 99.9%, or less than or equal to 100%.

The specific absorbent: CO₂ species molar ratios in the CO₂ rich and CO₂ lean solutions may be from as great as pure absorbent to as low as pure CO₂. It may be desirable for the CO₂ rich solution to comprise a greater molar ratio of absorbent:CO₂ species than the CO₂ lean solution. The CO₂ rich solution absorbent:CO₂ species molar ratios, include but are not limited to, less than 2:1, or less than 10:1 or any of the following: 8:1, or 6:1, or 4:1, or 2:1, or 1.9:1, or 1.85:1, or 1.8:1, or 1.75:1, or 1.7:1, or 1.65:1, 1.6:1, or 1.55:1, or 1.5:1, or 1.45:1, or 1.4:1, or 1.35:1, or 1.3:1, or 1.25:1, or 1.2:1, or 1.15:1, or 1.1:1, or 1.05:1 or 1:1, or 0.95:1, or 0.9:1. The CO₂ lean solution absorbent: CO₂ species molar ratios, include but are not limited to, greater than 1.5:1, or greater than any of the following: 100:1, or 50:1, or 10:1, or 8:1, or 6:1, or 4:1, or 2:1, or 1.95:1, or 1.9:1, or 1.85:1, or 1.8:1, or 1.75:1, or 1.7:1, or 1.65:1, 1.6:1, or 1.55:1, or 1.5:1, or 1.45:1, or 1.4:1, or 1.35:1, or 1.3:1, or 1.25:1, or 1.2:1, or 1.15:1, or 1.1:1, or 1.05:1 or 1:1, or 0.95:1, or 0.9:1.

### Soluble Substance Addition and Mixing Carbon Dioxide Desorption

The CO₂ rich solution enters the CO₂ desorption setup. The CO₂ rich solution may be a liquid solution or a liquid-solid slurry. In this step, a soluble substance and/or soluble substance containing solution is added to a CO₂ rich aqueous solution and CO₂(g) is subsequently desorbed, while the CO₂ absorbent, such as ammonia or an amine or other absorbents known in the art, predominantly remain in solution, such as less than 2% or less than any of the following: or 1%, or 0.5%, or 0.1% absorbent volatilization. The CO₂ desorption mechanism, may include, but is not limited to, the soluble substance interfering with the interactions between CO₂ species' and the CO₂ absorbent or CO₂ absorbents. Said interferences may include, but are not limited to, one or more or a combination of the following: reducing of solution dielectric constant, decrease in CO₂ species solubility, decrease in absorbent solubility, decrease in absorbent-CO₂ species compound solubility, decrease in absorbent-CO₂ species salt solubility, weakening of hydration shells surrounding dissolved CO₂ species, weakening of hydration shells surrounding CO₂ absorbent, weakening of hydration shells in absorbent-CO₂ species compound, weakening of hydration shells absorbent-CO₂ species salt, formation of a trimer, formation of an adduct, formation of a complex, formation of a complex ion, formation of a zwitterion, reaction with CO₂ absorbent, reversible reaction with CO₂ absorbent, reaction with CO₂ species, or reversible reaction with CO₂ species.

It may be desirable for the interaction of the soluble substance with the CO₂ absorbent-carbon dioxide salt to not involve a metathesis reaction or a single displacement reaction. It may be desirable for no chemical reaction to occur between the soluble substance and the CO₂ absorbent. It may be desirable for the CO₂ desorption to be entirely due to changes in solution media properties, such as changes in solution dielectric constant, changes in solution polarity, and changes in hydration shell stability.

The soluble substance may be preheated or cooled before injection into the mixing apparatus. The mixing apparatuses and methods include, but are not limited to, batch mixers, continuous stirred-tank reactors, CSTRs, distillation column, packed column, electrospray, spray column, countercurrent spray column, and/or other apparatuses and/or methods. The apparatus may be heated using waste heat or other heat source for, including, but not limited to, promoting CO₂ desorption, reducing viscosity and/or increasing the rate of solvent mixing.

The CO₂ may pressurize, by any means, including but not limited to, closing and opening a release valve to allow the system to pressurize, utilizing a smaller gas release valve, temperature change, or using external compression. In the case where the CO₂(g) is desorbed at a pressure greater than atmospheric pressure, less energy may be required for compression of this CO₂(g), if compression is desired. The exiting gas stream may contain predominantly CO₂. At least a portion of this desorbed CO₂ may be used for, including, but not limited to, one or more or a combination of the following: enhanced oil recovery, methanol production, syngas production, fuel production, urea production, fertilizer production, carbonate, bicarbonate production, carbamate production, beverage production, greenhouse, agricultural applications, welding gas, turbine working fluid, laser gas, food production, inert gas, cement production, CO₂ conversion processes, and other existing and future applications. This gas stream may be further treated by, including, but not limited to, water wash down, aqueous wash down, non-aqueous wash down, changes in pressure, changes in temperature, compression, vacuum, and an additional carbon capture process. Additives may be added to this gas stream prior, during or after treatment or in the absence of treatment. These additives include, but are not limited to, ammonia, electricity, light, hydrogen, amine, oxygen, methane, methanol, carbon monoxide, hydrogen sulfide, haloalkanes, chlormethane, dimethylether, hydrogen cynide, sulfur, acid or acid gas, hydroxide, oxide, carbonate, carbamate, and bicarbonate.

Maintaining CO₂(g) in Headspace: Measures may be taken to ensure the gas stream or headspace contains a high concentration of CO₂(g), especially during the first instance of use or after construction. This may be achieved by, including, but not limited to, purging the CO₂(g) generation vessel with pure CO₂(g) before the first run of the process. Self-purging may also be employed by using the CO₂(g) desorbed during solvent addition in initial runs to displace or dilute the other gases present in the vessel.

Added Solvent: The added soluble substance may include, but is not limited to, one or more or a combination of the following: organic solvents, concentrated soluble substance solutions, water soluble polymers, combinations of soluble substances, solvent mixtures, emulsions, pure substance, pure solvent, aqueous solvent, surfactant containing solvents, zwitterions, solids, soluble solids, gases, liquid-solid mixtures, soluble gases, aerosols, suspended solids, solid-gas mixtures, super critical fluids, and fluid mixtures.

Precipitate during Solvent Addition CO₂ Desorption: When a soluble substance is added to a CO₂ rich solution, such as 2M aqueous ammonium bicarbonate, in addition to the desorption of CO₂(g), a portion of the CO₂ containing salt may precipitate as a solid. This precipitate may dissolve back into solution, including, but not limited to, as CO₂(g) desorption occurs. This may be due to ammonium carbonate or carbamate (NH₃:CO₂ of 2:1) being more soluble than ammonium bicarbonate (NH₃:CO₂ of 1:1) in the water-soluble substance solution. In some embodiments there is no substantial precipitate formed.

Application of Heating or Cooling: Heating or cooling may be incorporated throughout the integrated process. For example, heating or cooling may be beneficial during CO₂ desorption to increase CO₂(g) yield and soluble substance solubility. Polyethylene glycols (PEGs) and polypropylene glycols (PPGs), for example, have higher Gibbs free energy of mixing and osmotic pressure at lower temperatures. Cooling may enhance CO₂(g) desorption, including, but not limited to, due to the greater Gibbs free energy of mixing and osmotic pressure of PEGs and PPGs at cooler temperatures and the decreased solubility of the CO₂ containing salts, such as ammonium bicarbonate or carbonate, at lower temperatures. Heating may enhance CO₂(g) desorption, including, but not limited to, due to greater reaction kinetics and lower CO₂ species solubility.

The soluble substance may be added to the CO₂ rich solution as a concentrated aqueous or non-aqueous solution or in a pure form. Said concentrated solution of the soluble substance may contain a vol/vol % concentration of soluble substance as low as 0.0001% to as great as 99.99999%. Vol/vol % concentrations of the soluble substance or concentrated soluble substance solution may be practically greater than any of the following: 1%, or 5%, or 10%, or 11%, or 12%, or 13%, or 14%, 15%, or 16%, or 17%, or 18%, or 19%, or 20%, or 21%, or 22%, or 23%, or 24%, or 25%, or 26%, or 27%, or 28%, or 29%, or 30%, or 31%, or 32%, or 33%, or 34%, or 35%, or 36%, or 37%, or 38%, or 39%, or 40%, or 41%, or 42%, or 43%, or 44%, or 45%, or 46%, or 47%, or 48%, or 49%, or 50%, or 51%, or 52%, or 53%, or 54%, or 55%, or 56%, or 57%, or 58%, or 59%, or 60%, or 65%, or 70%, or 75%, or 80%, or 85%, or 90%, or 95%, or less than or equal to 100%.

The resulting concentration of the soluble substance in the CO₂ desorption/mixing step may be a vol/vol % concentration of soluble substance as low as 0.0001% to as great as 99.99999%. Vol/vol % concentrations of the soluble substance in the CO2 desorption/mixing step or resulting mixed solution may be practically greater than any of the following: 0.1%, or 1%, or 2%, or 3%, or 4%, or 5%, or 5.5%, or 6%, or 6.5%, or 7%, or 7.5%, or 8%, or 8.5%, or 9%, or 9.5%, or 10%, or 10.5%, or 11%, or 11.5%, or 12%, or 12.5%, or 13%, or 13.5%, or 14%, or 14.5%, or 15%, or 15.5%, or 16%, or 16.5%, or 17%, or 17.5% or 18%, or 18.5%, or 19%, or 19.5%, or 20%, or 20.5%, or 21%, or 21.5%, or 22%, or 22.5%, or 23%, or 23.5% or 24%, or 24.5%, or 25%, or 25.5%, or 26%, or 26.5%, or 27%, or 27.5%, or 28%, or 28.5%, or 29%, or 29.5%, or 30%, or 31%, or 32%, or 33%, or 34%, or 35%, or 36%, or 37%, or 38%, or 39%, or 40%, or 41%, or 42%, or 43%, or 44%, or 45%, or 46%, or 47%, or 48%, or 49%, or 50%, or 51%, or 52%, or 53%, or 54%, or 55%, or 56%, or 57%, or 58%, or 59%, or 60%, or 65%, or 70%, or 75%, or 80%, or 85%, or 90%, or 95%, or less than 99%.

The maximum solubility of the soluble substance in the CO₂ desorption/mixing step may be a vol/vol % concentration of soluble substance as low as insoluble to as great as completely miscible. Vol/vol % solubility of the soluble substance may be practically greater than any of the following: 0.001%, 0.01%, 0.1%, or 1%, or 2%, or 3%, or 4%, or 5%, or 6%, or 7%, or 8%, or 9%, or 10%, or 11%, or 12%, or 13%, or 14%, or 15%, or 16%, or 17%, or 18%, or 19%, or 20%, or 21%, or 22%, or 23%, or 24%, or 25%, or 26%, or 27%, or 28%, or 29%, or 30%, or 31%, or 32%, or 33%, or 34%, or 35%, or 36%, or 37%, or 38%, or 39%, or 40%, or 41%, or 42%, or 43%, or 44%, or 45%, or 46%, or 47%, or 48%, or 49%, or 50%, or 51%, or 52%, or 53%, or 54%, or 55%, or 56%, or 57%, or 58%, or 59%, or 60%, or 61%, or 62%, or 63%, or 64%, or 65%, or 66%, or 67%, or 68%, or 69%, or 70%, or 71%, or 72%, or 73%, or 74%, or 75%, or 76%, or 77%, or 78%, or 79%, or 80%, or 81%, or 82%, or 83%, or 84%, or 85%, or 86%, or 87%, or 88%, or 89%, or 90%, or 90.5%, or 91%, or 91.5%, or 92%, or 92.5%, or 93%, or 93.5%, or 94%, or 94.5%, or 95%, or 95.5%, or 96%, or 96.5%, or 97%, or 97.5%, or 98%, or 98.5%, or 99%, or 99.5%, or 99.9%, or 100%, or completely miscible.

Purity of CO₂ Desorbed: The purity of CO₂ may desirably by greater than 90%. The CO₂ concentration range may be as low as 0.0001% to as great as 99.99999%. The purity or concentration of the desorbed CO₂ may be as low as any of the following: 0.1% or greater than 0.1%, or 1%, or 2%, or 3%, or 4%, or 5%, or 6%, or 7%, or 8%, or 9%, or 10%, or 11%, or 12%, or 13%, or 14%, or 15%, or 16%, or 17%, or 18%, or 19%, or 20%, or 21%, or 22%, or 23%, or 24%, or 25%, or 26%, or 27%, or 28%, or 29%, or 30%, or 31%, or 32%, or 33%, or 34%, or 35%, or 36%, or 37%, or 38%, or 39%, or 40%, or 41%, or 42%, or 43%, or 44%, or 45%, or 46%, or 47%, or 48%, or 49%, or 50%, or 51%, or 52%, or 53%, or 54%, or 55%, or 56%, or 57%, or 58%, or 59%, or 60%, or 61%, or 62%, or 63%, or 64%, or 65%, or 66%, or 67%, or 68%, or 69%, or 70%, or 71%, or 72%, or 73%, or 74%, or 75%, or 76%, or 77%, or 78%, or 79%, or 80%, or 81%, or 82%, or 83%, or 84%, or 85%, or 86%, or 87%, or 88%, or 89%, or 90%, or 90.5%, or 91%, or 91.5%, or 92%, or 92.5%, or 93%, or 93.5%, or 94%, or 94.5%, or 95%, or 95.5%, or 96%, or 96.5%, or 97%, or 97.5%, or 98%, or 98.5%, or 99%, or 99.5%, or 99.9%, or less than or equal to 100%.

Partial Pressure of CO₂ Desorbed: The partial pressure of CO₂ may be greater than 0.5 atm or 1 atm. The CO₂ partial pressure range may be as low as 0.001 atm to as great as 100,000 atm, liquid CO₂, supercritical CO₂, or solid CO₂. The partial pressure of CO₂ may be as low as any of the following: 0.001 atm, or 0.01 atm, or greater than or less than 0.05 atm, or 0.1 atm, or 0.2 atm, or 0.3 atm, or 0.4 atm, or 0.5 atm or 0.6 atm, or 0.7 atm, or 0.8 atm, or 0.9 atm, or 1 atm, or 1.1 atm, or 1.2 atm, or 1.3 atm, or 1.4 atm, or 1.5 atm, or 1.6 atm, or 1.7 atm, or 1.8 atm, or 1.9 atm, or 2 atm, or 2.1 atm, or 2.2 atm, or 2.3 atm, or 2.4 atm, or 2.5 atm, or 2.6 atm, or 2.7 atm, or 2.8 atm, or 2.9 atm, or 3 atm, or 3.5 atm, or 4 atm, or 4.5 atm, or 5 atm, or 5.5 atm, or 6 atm, or 6.5 atm, or 7 atm, or 7.5 atm, or 8 atm, or 8.5 atm, or 9 atm, or 9.5 atm, or 10 atm, or 12 atm, or 15 atm, or 18 atm, or 20 atm, or 22 atm, or 25 atm, or 28 atm, or 30 atm, or 40 atm, or 50 atm, or 60 atm, or 75 atm, or 100 atm, or 150 atm, or 200 atm, or 500 atm, or 1,000 atm, or 10,000 atm, or 100,000 atm, or less than 1,000,000 atm.

The purity or concentration of the desorbed CO₂ or final CO₂ produced may be dependent on the application. The setup may contain other gases than CO₂(g). The other gas or gases present in with this CO₂ may be dependent on the application. For example, if the CO₂ will be mixed with hydrogen (such as at about a 2:1 or 3:1 ratio) to produce CO₂ derived chemicals, hydrogen may be added as a headspace gas during CO₂ desorption. This example may reduce CO₂ capture energy requirements, including, but not limited to, due to the requirement of a lower partial pressure of CO₂(g) desorbed and lower final solvent concentration required.

### Soluble Substance and CO₂-Lean Absorption Solution Recovery

In this stage, the substance or substances may be recovered via one or more separation mechanisms. This stage involves separating the solution produced by the CO₂ desorption stage into two main streams: 1) the CO₂-lean absorption solution; 2) the soluble substance. The absorption solution is recycled back to the CO₂ absorption stage and the soluble substance is recycled back to the substance CO₂ desorption stage.

The separation devices and mechanisms employed are dependent on the type or types of added substances. Separation devices and mechanisms include, but are not limited to, one or more or a combination of the following: semi-permeable membrane, nanofiltration, organic solvent nanofiltration, reverse osmosis, ultrafiltration, microfiltration, hot nanofiltration, hot ultrafiltration, distillation, membrane distillation, flash distillation, multi-effect distillation, mechanical vapor compression distillation, switchable solvent, hybrid systems, thermally switchable solvent, centrifuge, or filter or combinations thereof.

Specific recovery methods or separation devices and mechanisms and combinations thereof are further described herein.

Nonvolatile Solvent Addition with Membrane Recovery

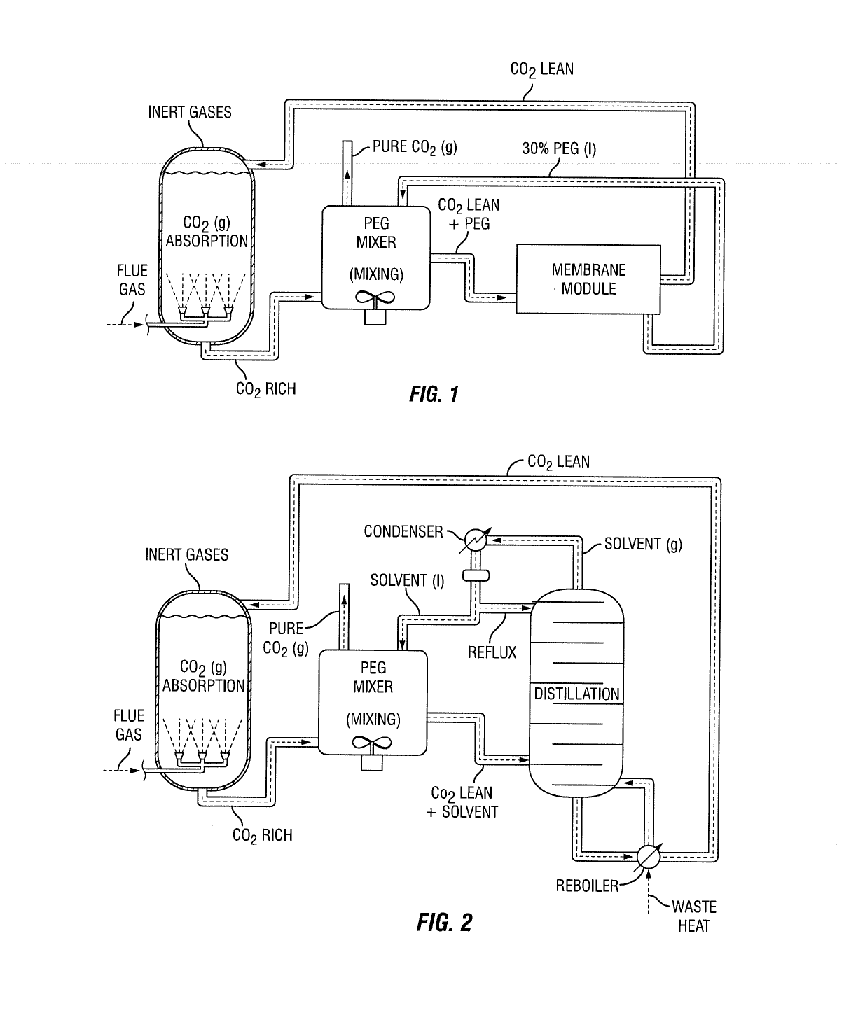

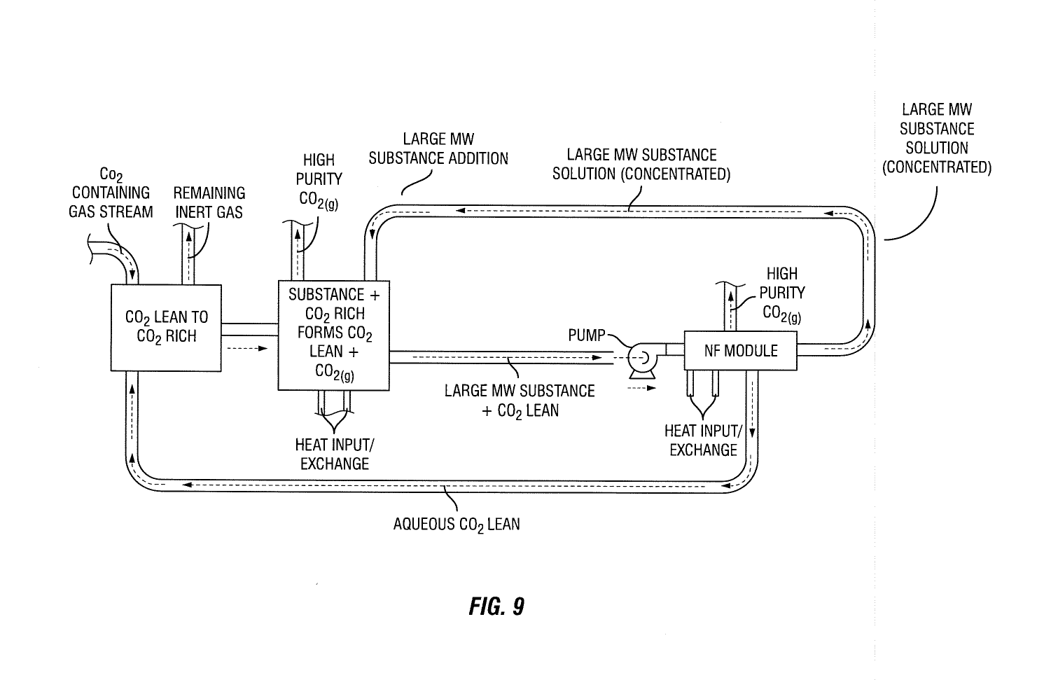

Example embodiments include, but are not limited to, FIG. 1. and FIG. 9.

### Embodiment End-to-End Overview:

1) Absorption: Gas containing CO₂ enters the absorption column and CO₂ is absorbed in a CO₂-lean aqueous absorbent-carbon dioxide solution, forming a CO₂-rich aqueous absorbent-solution. The remaining inert gases from the flue gas (N₂, O₂, Ar, low concentrations of CO₂) are released from the absorption column. The CO₂ rich solution created is transferred to stage 2.

2) CO₂ Desorption: A concentrated soluble substance solution or ‘concentrate’, such as 30% PEG, 70% CO₂ lean aqueous ammonia-carbon dioxide, is added and mixed with the CO₂ rich solution at a preset vol/vol % ratio under room temperature and pressure conditions. CO₂(g) is desorbed from the CO₂ rich solution and may undergo compression or other treatment prior to utilization. The CO₂-lean mixed solution containing the added substance, is transferred to stage 3.

3) Soluble Substance and Absorption Solution Regeneration via Nanofiltration: The CO₂-lean solution containing the soluble substance, such as Polyethylene Glycol (PEG) or Polypropylene Glycol (PPG), is fed into a nanofiltration membrane module and pressurized using a pump. The nanofiltration membrane rejects the dissolved soluble substance while allowing water, the CO₂ absorbent or absorbents (such as ammonia or amine) and carbon dioxide to pass. Two aqueous streams are generated: 1) a ‘concentrate’ stream (30% PEG(1) in diagram), which contains a high concentration of the soluble substance, such as PEG or PPG; 2) a ‘permeate’ stream (CO₂ lean in diagram) which contains less, minimal concentrations, or none of the added substance, such as PEG or PPG. Additional CO₂ may be desorbed from the ‘concentrate’ side and may be used as additional captured CO₂ (not shown in diagram). The ‘concentrate’ stream is transferred to stage 2 (CO₂ desorption) and the ‘permeate’ stream is transferred to stage 1 (flue gas CO₂ absorption).

Advantages, include, but are not limited to:
Completely new CO₂ desorption mechanism that is temperature independent.
   Capable of desorbing CO₂ at room temperature and pressure, or above or below room temperature
90% less energy required than most efficient existing amine processes
   Approaches thermodynamic limit for CO₂ capture
Highly Scalable—superior performance even at a small scale
No thermal input required
No significant retrofit to power plant
   Simply uses flue gas stream and a small amount of electricity (no steam or other thermal input)
Non-toxic, non-volatile and widely available reagents
Low Capital Cost
   Ultra-low cost and widely available reagents, including, but not limited to—soluble polymer (including, but not limited to, PEG, PPG, or other substances), Ammonia or other CO₂ absorbent, Water
   Inexpensive Materials—Room Temperature, standard industrial equipment (absorption column, mixer, wastewater treatment nanofiltration modules)
Minimal or no reagent degradation

Description: The embodiment is composed of three main steps: 1) The addition/contacting of a gas containing CO₂ to convert aqueous ammonia, ammonium or amine containing CO₂ lean solution to a CO₂ rich solution. The remaining inert gases may undergo further purification, treatment or compression; 2) The addition of a large molecular weight (MW) water soluble substance or substances to the CO₂ rich solution to desorb CO₂(g), creating a CO₂ lean solution+added substance+CO₂(g). This CO₂(g) stream may undergo further purification, treatment or compression; 3) The recovery of the added substance or substances using a separation mechanism. The CO₂ lean aqueous soluble substance-CO₂ absorbent-carbon dioxide solution formed in the second stage is fed into a membrane module and may be separated using pressurization. The separation mechanism may include, but is not limited to, one or more or combination of the following: microfiltration, ultrafiltration, nanofiltration organic solvent nanofiltration and reverse osmosis. The membrane rejects the organic solvent or soluble substance, while allowing the CO₂ lean aqueous ammonia-carbon dioxide salt to pass through the membrane. The solution that passes through the membrane, or the permeate stream, is then transferred to the CO₂ absorption column. The solution rejected by the membrane, which contains a higher concentration of the soluble substance, is recycled to the CO₂ desorption stage as the soluble substance containing solution.

The type of membrane or filter employed may be dictated by the molecular weight of the soluble substance added, which may be advantageously larger than the molecular weight cut-off of the membrane. The molecular weight cut-off of the membrane or filter may be sufficiently large to allow aqueous ammonia-carbon dioxide species to pass though or to be minimally rejected. The power source of the pump is not of particular importance, however it may be powered by electricity, pressure exchanger, turbocharger, hydraulic pressure, heat, pressure retarded osmosis, or forward osmosis.

Following the membrane or filter based separation, energy can be recovered by both or either the permeate (the absorption solution) and the concentrate (the soluble substance containing solution). These energy recovery devices are known in the art and include, but are not limited to, pressure exchangers and turbochargers.

The embodiment may be heated or cooled where advantageous. For example, the solvent addition and mixing step may be heated or cooled for various purposes, including, but not limited to, increasing CO₂(g) yield, decreasing timeframe of CO₂(g) generation, increasing solvent solubility, reducing energy consumption in the membrane or filtration module or a combination thereof. Energy consumption in the membrane or filtration module may be reduced from solution or module heating due to, but not limited to, the one or more of the following: 1) reduction of osmotic pressure (which decreases with increasing temperature in PEGs, PPGs and other water soluble polymers), reduction in concentration polarization, reduction in viscosity and change in solubility. Any portion of the process may be heated or cooled. Heat sources may include, but are not limited to, waste heat, power plant waste heat, steam, heat, pump or compressor waste heat, industrial process waste heat, steel waste heat, metal refining and production waste heat, paper mill waste heat, cement production waste heat, calcination waste heat, factory waste heat, petroleum refining waste heat, solar heat, solar pond, air conditioner waste heat, combustion heat, geothermal heat, ocean or water body thermal heat, stored heat, and CO₂(g) absorption solution heat. Temperatures of heating or cooling for any of the embodiments disclosed include, but are not limited to, less than any of the following: −20° C., or −10° C., or 0° C., or 10° C., or 20° C., or 25° C., or 30° C., or 35° C., or 40° C., or 41.5° C., or 41.5° C., or 41.5° C.-60° C., or 45° C., or 50° C., or 55° C., or 60° C., or 60-100° C., or 110° C., or 150° C. For example, power plant condenser waste heat is generally abundant at ˜41.5° C. and may be employed. Relatively lower molecular weight solvents may be employed if advantageous, including, but not limited to, polyethylene glycols 150-2000, polypropylene glycols 425-4000 and glycol ethers, such as triglyme. Although relatively lower molecular weight solvents or soluble substances, such as polyethylene glycols 150-2000, may have a higher osmotic pressure for a given volume/volume % concentration, these may be advantageous due to including, but not limited to, one or more of the following: 1) exhibit lower viscosity, 2) higher solubility, 3) less prone to degradation, 4) less expensive, 5) lower concentration polarization, 6) higher mole fraction per given vol/vol %, 7) greater Gibbs free energy of mixing and 8) greater influence on dielectric constant. Relatively larger molecular weight solvents may be advantageous due to one or more of the following: 1) lower osmotic pressure, 2) greater reduction of osmotic pressure with heat, 3) allow for the use of a larger pore size membrane or filter, 4) allow for the use of a higher permeability membrane, 4) may possess an LCST or UCST phase change with temperature and 5) may decrease in solubility with changes in temperature. The process may be constructed for large scale, stationary CO₂ capture.

The process may also be constructed and transported in smaller scale modules or as a unit, such as in shipping containers and transported and used in other locations. This may facilitate the ability to capture carbon dioxide in remote locations, in applications including, but not limited to, oil and gas production, cement production, mining and air CO₂ capture. The process may also be constructed as a stationary process.

The added concentrate, which may be a solution with a high concentration of the large molecular weight soluble substance, may comprise one or more or a combination of the following: a solid, a liquid, an aqueous solution containing the recovered substance, an aqueous solution containing the recovered solvent and CO₂ absorption species, an aqueous solution containing the recovered solvent and CO₂ absorption species and CO₂ species or a combination thereof.

Application of Heating or Cooling: Heating prior or during membrane recovery may reduce energy consumption due to, including, but not limited to, lower osmotic pressure and lower concentration polarization. Chilling may be useful in the absorption column to reduce ammonia slip.

If the solution is mildly heated, the energy required to separate water soluble polymers, hydrogels and other substances separable via a membrane may be reduced for reasons, including but not limited to, 1) lower concentration polarization; 2) lower viscosity; 3) lower osmotic pressures at higher temperatures in aqueous solutions. CO₂(g) may be desorbed during Step 3 with or without heating. This CO₂(g) and other CO₂(g) desorbed at or between stages 1, 2 or 3 of this process may undergo the same use or treatment as the CO₂(g) desorbed from the desorption stage (stage 2), including use as captured CO₂(g). CO₂(g) may be desorbed due to, including, but not limited to, one or more or a combination of the following: increase in soluble substance concentration, a further decrease in the dielectric constant in the solution, weakening of the hydration shells solvating the aqueous ammonia (or other CO₂ absorbent molecule or molecule combination)-carbon dioxide compound, or changes in temperature or pressure. The pressure of the CO₂(g) generated may supplement the pressurization energy requirements of the pump or other pressurization method.

The regeneration portion of this embodiment may employ, including, but not limited to one, or more or a combination of the following: reverse osmosis, nanofiltration, organic solvent nanofiltration, ultrafiltration, microfiltration or switchable solvent.

The embodiment may employ a reverse osmosis membrane with a low molecular weight cut-off, including but not limited to, less than any of the following: 250 da, or 200 da, or 150 da, or 125 da, or 100 da, or 95 da, or 90 da, or 85 da, or 80 da, or 75 da, or less than the hydration radius of ammonium bicarbonate. For example, this embodiment may employ aqueous ammonia as the CO₂ absorbent. In this instance, it may be advantageous for the NH₃:CO₂ molar ratio of the aqueous ammonia-carbon dioxide in the solution produced by the desorption stage to be greater than 1.5:1 or the pH to be greater than about 8.5. Ionic aqueous ammonia (or ammonium)-carbon dioxide species may become free dissolved ammonia or carbon dioxide under these conditions. The hydration radius of free ammonia or carbon dioxide is significantly smaller than the hydration radius of ionic species of ammonia (ammonium) and carbon dioxide (bicarbonate or carbonate or carbamate). Thus, under these conditions, the ammonia-carbon dioxide may more freely pass through a relatively small molecular weight cut-off reverse osmosis or forward osmosis membrane. This may allow for the use of lower molecular weight added substances, such as ethylene glycol, ethylene carbonate, propylene glycol, propylene carbonate, and polyethylene glycol (PEG) 200, which may be advantageous due to, including, but not limited to, one or more or a combination of the following: greater solubility, lower viscosity, lower cost, exhibit a greater Gibbs free energy of mixing, exhibit a greater influence on solution dielectric constant, less prone to degradation, and exhibit less concentration polarization during membrane solvent recovery. Additionally, it may allow for appreciably complete recovery or removal of the added solvent, including when a relatively larger molecular substance is employed.

Multicomponent separation devices or multistage separation devices may be employed. Said device or devices may include, but are not limited to, one or more or a combination of the following: binary distillation, azeotrope distillation, membrane distillation, mechanical vapor compression, hybrid systems, flash distillation, multistage flash distillation, multieffect distillation, extractive distillation, switchable solvent, reverse osmosis, nanofiltration, organic solvent nanofiltration, ultrafiltration, and microfiltration. For example, such a hybrid system may involve at least partially recovering the soluble substance using nanofiltration and then further concentrating the soluble substance using membrane distillation.

Another example of such a hybrid system may be a process wherein a switchable solvent ‘switches’ out of solution due to the presence of a stimulant, such as a change in temperature, then nanofiltration is employed to further concentrate the switchable solvent or remove remaining switchable solvent in the CO₂ lean solution. The switchable solvent or other substance dissolved in solution may be further recovered or concentrated or even removed from the one or more layers or separate solutions that are formed.

Applied Pressure or Osmotic Pressure of Solution: The osmotic pressure range of the resulting water soluble substance solution may be as low as 0.001 atm to as great as 1,000,000 atm. The osmotic pressure may be as low as less than any of the following: 0.001 atm, or 0.01 atm, or greater than or less than 0.05 atm, or 0.1 atm, or 0.2 atm, or 0.3 atm, or 0.4 atm, or 0.5 atm or 0.6 atm, or 0.7 atm, or 0.8 atm, or 0.9 atm, or 1 atm, or 1.1 atm, or 1.2 atm, or 1.3 atm, or 1.4 atm, or 1.5 atm, or 1.6 atm, or 1.7 atm, or 1.8 atm, or 1.9 atm, or 2 atm, or 2.1 atm, or 2.2 atm, or 2.3 atm, or 2.4 atm, or 2.5 atm, or 2.6 atm, or 2.7 atm, or 2.8 atm, or 2.9 atm, or 3 atm, or 3.5 atm, or 4 atm, or 4.5 atm, or 5 atm, or 5.5 atm, or 6 atm, or 6.5 atm, or 7 atm, or 7.5 atm, or 8 atm, or 8.5 atm, or 9 atm, or 9.5 atm, or 10 atm, or 12 atm, or 15 atm, or 18 atm, or 20 atm, or 22 atm, or 25 atm, or 28 atm, or 30 atm, or 35 atm, or 40 atm, or 45 atm, or 50 atm, or 55 atm, or 60 atm, or 65 atm, or 70 atm, or 75 atm, or 80 atm, or 85 atm, or 90 atm, or 95 atm, or 100 atm, or 150 atm, or 200 atm, or 500 atm, or 1,000 atm, or 10,000 atm, or 100,000 atm, or less than 1,000,000 atm, or pure solvent.

### Science:

Organic Solvent and CO₂ Lean Absorbing Solution Recovery:

This embodiment employs a nanofiltration membrane with a pore size sufficiently small to reject the large molecular weight organic solvent and sufficiently large to allow aqueous ammonia-carbon dioxide salts to pass through the membrane. An effective membrane for this process may have molecular weight cutoff of above 200 Daltons to allow hydrated ammonia or amine and carbon dioxide to pass through the membrane and below the molecular weight of the organic solvent or soluble substance, such as PEG 600.

Energy for separation is supplied by pressurization, which may be accomplished using electricity and pumps used in commercial reverse osmosis desalination and nanofiltration process. Energy requirements in commercial aqueous membrane-based separation processes can approach the minimum thermodynamic energy requirement, exponentially improving the efficiency of CO₂ capture. Embodiments may include:
Desorbing CO₂ through the presence of a water-soluble solvent and end-to-end process
Using non-volatile solvents, including, but not limited to, Polyethylene glycols
Separating water soluble solvents using semipermeable membrane, including, but not limited to, nanofiltration membrane
   Solvent(s) at least partially rejected by membrane(s)
   Absorption solution (ammonia, water, CO₂) is not rejected or minimally rejected by said membrane(s)
Using waste heat or chilling to accelerate or foster CO₂ desorption and other hybrid waste heat and membrane recovery process combinations

### Experimental Data

Embodiment Tested: (1) CO₂ is absorbed in the CO₂ lean aqueous ammonia in the absorption column, forming CO₂ rich aqueous ammonia; (2) PEG concentrate is added and mixed with the CO₂ rich aqueous ammonia, desorbing CO₂ and forming a CO₂ lean solution; (3) PEG concentrate and CO₂ lean aqueous ammonia are separated using nanofiltration and recycled. Nanofiltration membranes reject PEG, while ammonia, water and carbon dioxide species pass through the membranes.

CO₂ Desorption Experiments: The CO₂ desorption stage of this embodiment involves adding the substance, such as a concentrated aqueous PEG solution, to the CO₂ rich aqueous CO₂ absorbent-carbon dioxide solution, such as ammonia-carbon dioxide, from the absorption column. Pure CO₂(g) is desorbed at room temperature and pressure (RTP) conditions.

The following shows data from experiments assessing the desorption rate and total yield of pure CO₂(g) in relation to final PEG concentration and ammonium bicarbonate concentration. Pure PEG 600 was injected and mixed at a consistent mixing rate. The CO₂ desorption trials were conducted at room temperature and atmospheric pressure.

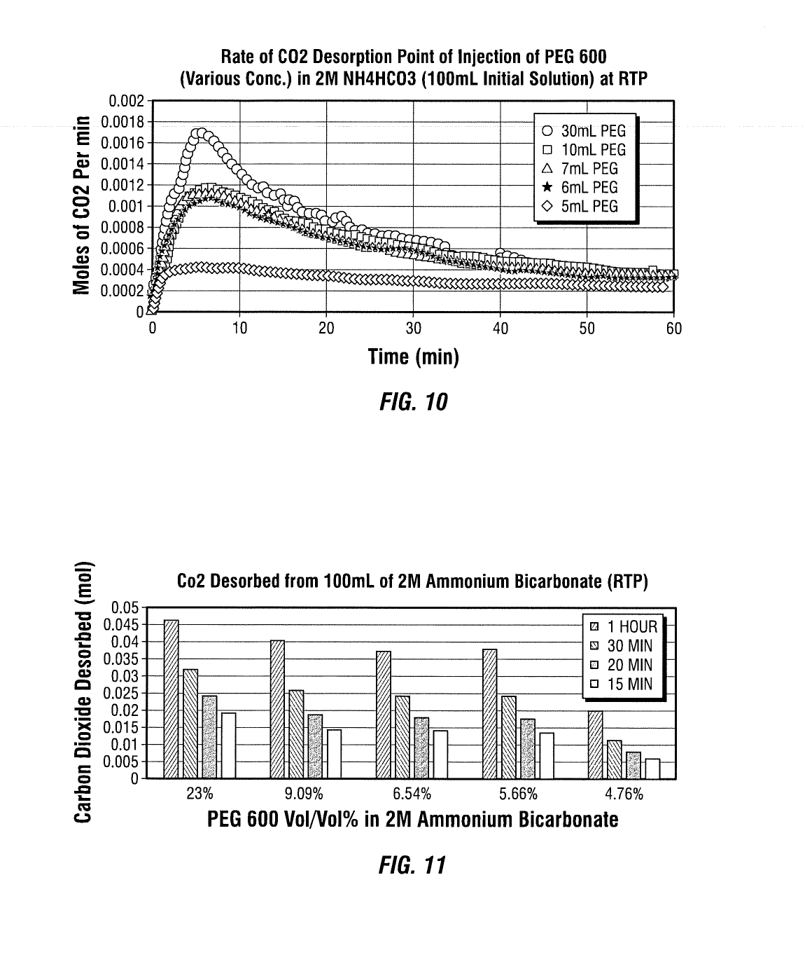

The graph at FIG. 10 shows the rate of CO₂ generation in relation to final PEG concentration.

### Note:

30 mL=30 mL PEG+100 mL 2M NH₄HCO₃(aq)=˜23% PEG
10 mL=10 mL PEG+100 mL 2M NH₄HCO₃(aq)=9.09% PEG
7 mL=7 mL PEG+100 mL 2M NH₄HCO₃(aq)=6.54% PEG
6 mL=6 mL PEG+100 mL 2M NH₄HCO₃(aq)=5.66% PEG
5 mL=5 mL PEG+100 mL 2M NH₄HCO₃(aq)=4.76% PEG
The graph at FIG. 11 shows the total CO₂ generation in respect to final PEG concentration and time.
While not wishing to be bound by any particular theory it may be concluded:
1. There is a threshold concentration to initiate CO₂ desorption. In the case of PEG 600 (graph above), this threshold concentration is ˜6 mL or 5.66% concentration. Anything below this concentration will minimally desorb CO₂ (as seen by the 5 mL case).
2. Increasing the concentration beyond this threshold concentration will result in greater CO₂ desorption. The influence of increasing the solvent concentration on total CO₂ desorption and rate of CO₂ desorption diminishes the higher the concentration.
3. CO₂ desorption rates are greatest during the first 15-20 minutes after solvent addition.

Optimization may involve, including, but not limited to, changing mixing rate, soluble substance type, soluble substance concentration, CO₂ absorbent solution, CO₂ absorbent concentration, CO₂ absorbent combination and temperature. Optimization of solvent type may involve determining the most effective molecular weights and molecular structures of each soluble substance type and making most effective use of each soluble substances' properties. For example, the osmotic pressure of aqueous propylene glycols (PPGs) decrease significantly with temperature, even becoming thermally switchable at higher temperatures. For example, the osmotic pressure of a 50% PPG 425 vol/vol solution is ˜75% less at 40° C. than 20° C. (pg. 38, http://projekter.aau.dk/projekter/files/17652274/Investigation_of_Polypropylene_Glycol_425_as_a_Draw_Solution_for_Forward_Osmosis.pdf, incorporated herein by reference). An example of making optimal use of this property may involve preheating the solution produced in the CO₂ desorption mixer prior to the nanofiltration soluble substance recovery stage. This may reduce regeneration energy requirements by reducing osmotic pressure, viscosity and concentration polarization.

These experiments use 2M concentration ammonium bicarbonate, which is near the ammonium bicarbonate solubility limit of 2.7M at 20° C. Higher concentrations than 2.7M may be employed, even at temperatures below 20° C., as the process may function properly under conditions where the CO₂ rich or CO₂ lean streams comprise of solids or contain solids or comprise a solid-liquid slurry.

Solid precipitation and dissolution may occur throughout the process, including, but not limited to, due to changes in soluble substance concentration, CO₂ loading, and temperature. For example, precipitate may form in the absorption column, such as due to the increase in CO₂ concentration. The precipitate may dissolve back into solution during the CO₂ desorption or soluble substance regeneration stages, due to, including, but not limited to, a decrease in CO₂ concentration, increases in temperature, and recovery of at least a portion of the soluble substance.

Prior to the regeneration stage, such as nanofiltration soluble substance recovery or distillation soluble substance recovery, a separation device, such as a filter, may be employed, for purposes, including but limited to, preventing the buildup of solids in the substance regeneration component.

Solubility of ammonium bicarbonate increases with temperature until it begins to decompose at 40° C.-60° C. Thus, higher concentrations than 2.7M (the solubility limit of ammonium bicarbonate in water at 20° C.) may be still contain no solids, including, but not limited to, if the temperature is raised. In this instance, ammonium bicarbonate, carbonate or sesquicarbonate precipitate may form in the absorption column, including, but not limited to, because the absorption column may operate at near or below room temperature to prevent ammonia slip. Maximizing concentration may be useful as it may, including, but not limited to, increase the CO₂ absorption-desorption capacity.

### Calculations

Nanofiltration PEG 600 Energy Requirement Calculations: This stage employs nanofiltration membranes with a pore size sufficiently small to reject the large molecular weight organic solvent, such as polyethylene glycol, or other soluble substance and sufficiently large to allow aqueous ammonia-carbon dioxide salts, or other CO₂ absorbent-CO₂ species, to pass through the membrane. An effective membrane for this process may have a molecular weight cutoff of above 200 Daltons to allow hydrated ammonia and carbon dioxide to pass through the membrane and below the molecular weight of the organic solvent (e.g. PEG 600).

Aqueous PEGs are commonly employed to evaluate the molecular weight cutoff of standard reverse osmosis and nanofiltration membranes. PEG is nontoxic and inert, and may pose little threat of degradation, fouling or other unintended interaction with nanofiltration membranes. The process may use standard industrial nanofiltration membrane modules and setups known in the art.

Energy for separation may be supplied by pressurization, which may be accomplished using electricity and pumps used in commercial reverse osmosis desalination and nanofiltration processes known in the art. Energy requirements in commercial aqueous membrane-based separation processes may approach the minimum thermodynamic energy requirement, exponentially improving the efficiency of CO₂ capture.

Below is the modelled energy consumption of the process and estimated the CO₂ generated from a 9.09% v/v concentration solution of PEG 600 in a CO₂ rich aqueous ammonia-carbon dioxide solution. 9.09% v/v was a sufficient concentration of PEG 600 to convert 25% of aqueous CO₂ into pure CO_{2(g)} over a 30-minute period. The energy consumption per kg of CO₂ captured is slightly greater than the thermodynamic limit for flue gas capture (172 kJ/kg CO₂) and over 96% less energy than the chilled ammonia process.

| | |
|---|---|
| Pressure | |
| (1.5*π) | Energy Consumption |

| ATM | (MJ/m\hat{ }3) | (MJ/kg of CO₂ captured) |
|---|---|---|
| 48.5 | 4.9 | 0.222 |

The nanofiltration setup may be designed based on optimized solution flow rates and PEG concentration in the ‘PEG-concentrate’ added solution and the mixed solution. These parameters may be determined based on the absorption column and CO₂ desorption stages.

### Types of Substances

Generally desirable properties: There are a wide range of substances capable of being added to an aqueous solution containing ammonia, ammonium, amine or bicarbonate, carbonate or carbamate species that would desorb CO₂ can be subsequently recovered using membrane or filter based processes (e.g. Microfiltration, Ultrafiltration, Nanofiltration, Reverse Osmosis). The following is a list of potentially desirable properties for these added substances. Desired substances may include one or more of the following, although the properties are not limited to those described herein and added substances may or may not exhibit any of these properties.
High solubility in water or aqueous solutions
Molecular weight sufficient in size to be rejected by the desired membrane or filter (above the molecular weight cut-off)
Low cost
Non-hazardous and compatible with most conventional equipment
Does not react with ammonia or carbon dioxide in unfavorable ways
Does not degrade or degrades slowly or degradation can be inhibited

Solvents that meet the properties thereof include, but are not limited to, a wide range of glycols (such as polyethylene glycols [PEG] and polypropylene glycols [PPG]).

Solvent Addition with Distillation Recovery

Embodiments described include the embodiment shown in FIG. 2.

Brief Description: The system is composed of three main steps: 1) Gas containing CO₂ enters the absorption column and CO₂ is absorbed in a CO₂-lean aqueous absorbent-carbon dioxide solution, forming a CO₂-rich aqueous absorbent-carbon dioxide solution. The remaining inert gases from the flue gas (N₂, O₂, Ar, low concentrations of CO₂) are released from the absorption column. 2) The addition of a water-soluble solvent to a CO₂ rich solution, resulting in the formation of gaseous CO₂(g) and a CO₂-lean solution. The gaseous CO₂(g) may undergo further purification or treatment to remove solvent, water vapors, or traces of absorbent vapor, which may be recycled in the process; 3) The distillation and condensation of the low boiling point solvent from the remaining CO₂ lean solution, which may include using ultra low grade heat (less than any of the following: ˜42° C., or 60° C., or 80° C., or 100° C.). The CO₂ lean solution, which now contains an appreciably lower concentration of organic solvent, is circulated to the absorption column, while the condensed organic solvent is circulated to the substance addition desorption stage.

In instances where the solvent used has a higher boiling point than the temperature of the waste heat, distillation may be conducted by exploiting the high vapor pressure of the solvent via one or more or a combination of the following: multi-effect distillation, membrane distillation, a lower temperature condenser, vapor compression, or mechanical vapor compression distillation. The CO₂-lean solution, after the recovery of the solvent, may be recycled to the first step of the process.

1) Absorption:

Dilute CO₂ in flue gas is absorbed in a CO₂ lean aqueous ammonia-carbon dioxide solution forming a CO₂ rich aqueous ammonia-carbon dioxide solution. The CO₂(g) lean aqueous ammonia-carbon dioxide solution may be composed of predominantly aqueous ammonium carbonate and ammonium carbamate at an NH₃:CO₂ molar ratio that may be greater than 1.5:1 and may be near 2:1. CO₂(g) is absorbed in the CO₂ lean aqueous ammonia to form aqueous ammonium bicarbonate at an NH₃:CO₂ molar ratio, such as less than 1.5:1 and near 1:1. Dilute CO₂(g) is absorbed in a CO₂ lean aqueous ammonia-carbon dioxide solution according to the following chemical reaction:

(NH₄)₂CO₃(aq)+H₂O(aq)+CO₂(dilute gas)→2NH₄HCO₃(aq)

### 2) CO₂ Desorption:

CO₂ is desorbed by adding one or more water soluble, low cost organic solvents under moderate conditions to the CO₂ rich aqueous ammonia-carbon dioxide solution, such as room temperature and pressure conditions. In the case of the low-grade waste heat powered CO₂ capture process, a low boiling point organic solvent, such as acetone, dimethoxymethane, acetaldehyde, methyl formate, or dimethyl ether is employed. CO₂(g) is desorbed under substantially room temperature and pressure (RTP) conditions according to the following chemical reaction:

2NH₄HCO₃(aq)+Organic Solvent(l)→(NH₄)₂CO₃(aq)+CO₂(g)+H₂O(aq)+Organic Solvent(aq)

CO₂(g) is desorbed from solution due to the organic solvent reducing the solution dielectric constant. It may be theorized that aqueous ammonia catalyzes and fosters the hydration of CO₂ into carbonic acid, thus enabling CO₂ to dissolve at a significantly greater concentration than it would without the presence of ammonia. The addition of an organic solvent may weaken the aqueous ammonia catalyzed hydration shells surrounding the dissolved CO₂ due to reduction of the solution dielectric constant, thus prompting the generation of CO₂(g) owing to the significantly lower solubility of aqueous phase CO₂ when uncatalyzed by ammonia. Significant pure CO₂(g) yields were achieved under room temperature and pressure conditions in a relatively short timeframe.

3) CO₂ Lean Absorbing Solution and Organic Solvent Recovery:

The CO₂ lean aqueous ammonia-carbon dioxide-organic solvent solution created in stage 2 is separated into pure organic solvent and an aqueous CO₂ lean ammonia-carbon dioxide solution using low temperature distillation. The CO₂ lean absorbent-carbon dioxide salt may predominantly remain in the aqueous solution during distillation, such as with absorbent volatilization of less than any of the following: 10%, or 5%, or 2%, or 1%, or 0.5%, or 0.1%, or 0.01%, while the organic solvent is volatized and condensed. The CO₂ lean absorbing solution and organic solvent are recovered according to the following:

(NH₄)₂CO₃(aq)+Organic Solvent(aq)+Low Grade Heat→(NH₄)₂CO₃(aq)+Organic Solvent(g)

Organic Solvent(g)+Condenser→Organic Solvent(l)

In this embodiment, it may be desirable for ammonia-carbon dioxide decomposition to be minimized.

CO₂ desorption or absorbent-CO₂ salt decomposition, unintended or intended, may occur during this stage. Desorbed CO₂ may be separated from the organic solvent vapor and treated similarly to the captured CO₂ produced in the desorption or mixing step. In the instance where the CO₂ absorbent exhibits volatility, such as in the case of ammonia, the CO₂ absorbent may be recycled, including, but not limited to, by dissolving in the added organic solvent or other added substance in the CO₂ desorption step.

Additional Details:
A multi-substance solvent may be used. Said solutions or mixtures may be desired to be azeotropes due to their property to function with a uniform boiling point. However, solvent mixtures do not have to be azeotropes, and may be mixtures of solvents that may or may not each boil at different temperatures. Mixtures may be composed of a combination of substances for any one or more reasons that may include, but are not limited to, improving properties, such as lower temperature boiling point, lower enthalpy of vaporization, greater solubility and lower dielectric constant or a solvent may be added to prevent an unfavorable reaction between the CO₂ absorbent salt and a substance.
CO₂ may be desorbed during the distillation step. This CO₂ and other gases that may be present, including, but not limited to, CO₂ absorbent, solvent vapor, and water vapor, may be separated and/or treated. CO₂ released in the distillation column and any other stage of the process may be utilized or treated by any methods or means, including those described for Stage 2.
The particular mechanism used to separate the added solvent from the solution may include, but is not limited to, one or more or a combination of the following: binary distillation, azeotrope distillation, mechanical vapor compression, membrane distillation, hybrid systems, flash distillation, multistage flash distillation, multieffect distillation, extractive distillation, switchable solvent, reverse osmosis, nanofiltration, organic solvent nanofiltration, ultrafiltration, and microfiltration.
The CO₂ desorption stage, the headspace gases may self-pressurize or pressurize. This may be advantageous due to, including, but not limited to, reductions in compression energy requirements and less energy demands for water wash down or other organic solvent and CO₂ absorbent separation process.
Water wash-downs or other treatment processes may be applied at any stage of the process, including to some or all entering and exiting fluid streams. This includes, but is not limited to:
   Purification or removal of one or more or a combination of the following from the gas stream exiting the CO₂ absorber or ‘inert gases: organic solvent, ammonia, other CO₂ absorbent, other impurity, other chemical or water
   Purification or removal of one or more or a combination of the following from the gas stream exiting the CO₂ desorption stage: organic solvent, ammonia, other CO₂ absorbent, other impurity, other chemical or water
   Purification or removal of one or more or a combination of the following from the gas stream, if any, exiting the soluble substance recovery stage: organic solvent, ammonia, other CO₂ absorbent, other impurity, other chemical or water
Larger molar mass water soluble molecules, such as soluble molecules with a molecular weights greater than 200 daltons, may be included in solution to reduce total quantity of moles and increase the added organic solvent mole fraction, which may reduce temperature requirements during distillation in accordance with Raoult's Law.

For the embodiment shown in FIG. 2, the solvent may be desired to possess, including, but not limited to, a low boiling point, low dielectric constant, low enthalpy of vaporization, no azeotrope with water (or an azeotrope with a higher mole fraction of the added solvent than water), low toxicity and high solubility in water. Many solvents with favorable properties, may react or interact with the CO₂ absorbent in potentially unfavorable ways within the process unless additional measures are taken. Solvents that have a greater likelihood of reaction with ammonia or ammonium salts include those in the categories of Amines, Ketones, Aldehydes, Esters, and Carboxylic Acids. If solvents are used from these categories, it may be desirable for them to, include, but not be limited to, react to form a useful chemical, react slowly, react reversibly, or not react at all with CO₂ absorbents and CO₂ absorbent containing compounds. For example, acetone is a ketone, however its reaction with Ammonia sometimes requires months of continuous contact time, which may be unlikely or undesirable in the system. If solvents do react with Ammonia, other substances may be added to prevent an unfavorable reaction. For example, Methyl Formate, a solvent with a very low boiling point, high water solubility and low dielectric constant, reacts with ammonia to form formamide (an acid amide) and methanol. If Methanol is added to Methyl Formate, however, this may inhibit the forward reaction, and may allow Methyl Formate to be used as a solvent while inhibiting the reaction with ammonia. Additionally, Methyl Formate does form an azeotrope with Methanol, http://pubs.acs.org/doi/abs/10.1021/je200140m incorporated herein by reference. Therefore, the two solvents, if at the appropriate ratio to form the azeotrope, may boil at a uniform temperature.

Solvents that typically do not react with ammonia include those in the categories of Ethers and low molecular weight Alcohols. These substances rarely form unfavorable reactions with ammonia.

### Power Plant Waste Heat Quality and Quantity:

According to http://pubs.acs.org/doi/abs/10.1021/es5060989, incorporated herein by reference, 96% of power plant waste heat energy is at or below a temperature of 41.5° C. The remaining 4% is the higher temperature heat of the exhaust gas stream(s). Present carbon capture systems use higher temperatures (usually 110° C.-130° C.) and higher pressures (2-136 atm) and, therefore, cannot be solely powered by waste heat. Instead, these processes divert steam away from power generation and use it to power carbon capture. The process described herein may be capable of using abundant low temperature waste heat to capture carbon dioxide, allowing for the capture of carbon dioxide without appreciably impacting power plant efficiency.

### The Ammonia-Carbon Dioxide-Water System:

In this embodiment, the solvent may be distilled at or below 85° C. without appreciable volatilization of ammonia from solution at atmospheric pressure. In the instance where CO₂(g) or NH₃(g) are released from solution, these may re-dissolve in solution when the solvent is recycled. In the instance described thereof, CO₂(g) may be released in significant excess to NH₃(g), and may be removed as captured CO₂(g).

### Raoult's Law:

Raoult's law may be useful for the solvent distillation step of this embodiment. Raoult's Law describes the relationship between the mole fraction of a liquid in solution and the liquid's vapor pressure (ex. Mole fraction*Partial pressure of liquid at temperature=Partial pressure in system). In accordance with Raoult's Law, traces of added solvent may continue to remain in the CO₂ absorption solution. In an instance where traces of solvent have an influence on system performance, the timeframe of distillation may need to be lengthened. Distillation may be optimized to minimize energy demand, while achieving optimal solvent concentrations in the CO₂ absorber and CO₂ desorber.

### Experimental Data Volatile Organic Solvent Addition

Summary:

In these experiments, high purity CO₂ is generated through the addition of an organic solvent—such as acetone, dimethoxymethane, or acetaldehyde—to CO₂ rich, aqueous ammonia-carbon dioxide solution under room temperature and pressure conditions. The organic solvent and CO₂ absorbing solution are then regenerated using low temperature heat. When acetone, dimethoxymethane, or acetaldehyde were added at 16.7% (v/v) to 2 M aqueous ammonium bicarbonate, 39.8%, 48.6%, and 86.5%, respectively, of the aqueous CO₂ species transformed into high purity CO₂ gas over 3 hours. Thermal energy and temperature requirements to recover acetaldehyde and the CO₂ absorbing solution were 1.39 MJ per kilogram of CO₂ generated and 68° C., respectively, 75% less energy and 53° C. lower temperature than a pilot chilled ammonia process. These findings exhibit the promise of economically viable carbon capture powered entirely by abundant low temperature waste heat.

This embodiment generates high purity CO₂ via the addition of a water soluble organic solvent to a CO₂ rich aqueous ammonia-carbon dioxide solution, such as would be generated from the absorption of flue gas CO₂ in aqueous ammonia. The organic solvent is subsequently distilled using low temperature heat, resulting in recovery of the solvent and remaining CO₂ lean aqueous ammonia-carbon dioxide solution. Pure CO₂ is desorbed under room temperature pressure (RTP) conditions and employs only low cost, abundant reagents. The results demonstrate that this embodiment is capable of converting CO₂ in flue gas into high purity CO₂ with significantly lower temperature and energy requirements than current technologies.

Materials and Methods:

Measurements were acquired using a gas flow setup with on-line mass spectrometry. An Omega mass flow controller was used to control the flow rate of the carrier gas (ultra-high purity helium, 50 mL/min). The outlet line was heated to prevent solvent condensation. For each experiment, an appropriate amount of ammonium bicarbonate (>99.5%, Sigma Aldrich) was dissolved in deionized (DI) water to form 100 mL of total solution at a desired molarity (1.0, 1.5 M, or 2.0 M). A 250 mL glass media bottle containing the solution was attached to a three-port cap containing helium carrier gas inflow port, gas mixture outlet port, and organic solvent injection port. Helium gas was flowed into the headspace at 50 mL/min until no traces of air gases (N₂, O₂, and Ar) were present and the flow stabilized. Stirring was applied at a consistent mixing rate for all trials. An appropriate amount of organic solvent-acetaldehyde (≧99.5%, Sigma Aldrich), acetone (≧99.5%, Fisher Scientific), or dimethoxymethane (99%, Sigma Aldrich)—was injected. These solvents were selected based on their high solubility in water, low molar mass, low toxicity, high volatility relative to water, and lack of irreversible reactivity with ammonia or CO₂. A needle valve connected to a vacuum chamber with an SRS 100 residual gas analyzer was used to sample the outlet gas and obtain the CO₂ partial pressure. CO₂ partial pressures were converted to molar flow rates using a calibration curve derived from previous measurements of mass flow controlled ultrahigh purity CO₂ and by normalizing the signal intensity to the helium carrier gas. Integration of CO₂ flow rates over one hour yielded the values for total pure CO₂ generation.

Total CO₂ generation was determined by extrapolating results from 1 hour experiments with an ExpConvExp fitting function using the Multi-peak Fit package in Igor Pro (WaveMetrics Inc.). During long timeframe experiments with 20 mL of organic solvent added, CO2 generation tapered off after a three-hour period. Correspondingly, CO2 generation from 1 hour experiments were extrapolated to three hours. Three-hour extrapolations deviated less than 12.5% from experimental results.

Modeling Organic Solvent Distillation:

The heat duty and temperature requirements for the recovery of acetaldehyde, acetone, or dimethoxymethane from their respective aqueous solutions were determined using an industrial process modeling software (Aspen HYSYS) with the UNIQUAC fluid package. The simulation used a 1.5 m diameter distillation column with 10 sieve trays and a 0.1 m³ reboiler and condenser. The feed stream flow rate was 1 m³ solution per hour and contained the optimal organic solvent mole fraction (x_{f}) to generate pure CO₂ (0.0467 for acetone, 0.0393 for dimethoxymethane, and 0.0599 for acetaldehyde) in water. According to vapor—liquid equilibrium studies, only slight traces of NH₃ and CO₂ vaporize at the low temperatures employed. The simulated distillation column was at a scale sufficient for 0.43-1.83 tons CO₂ captured per day, or a similar scale to the referenced current process pilot plants, 1.7 and 4.0 tons of CO₂ per day for chilled ammonia and MEA, respectively. The optimal organic solvent mole fraction was experimentally determined by adding the organic solvent at RTP conditions to a 100 mL 2 M aqueous ammonium bicarbonate solution until the injection of additional organic solvent had no discernable influence on CO₂ generation. The feed solution was distilled to the operational organic solvent mole fraction (x_{b}) in the regenerated solution (0.0216 for acetone, 0.0181 for dimethoxymethane, and 0.0279 for acetaldehyde). The operational organic solvent mole fraction was experimentally determined by adding small amounts of organic solvent at RTP conditions to a 100 mL 2 M aqueous ammonium bicarbonate solution until the injection of additional organic solvent resulted in CO₂ generation (FIG. S2).

### Results and Discussion

System Overview: This embodiment is composed of three steps: (1) flue gas CO₂ absorption in CO₂ lean aqueous ammonia solution, (2) pure CO₂ generation through mixing in an organic solvent, and (3) recovery of organic solvent via low-temperature distillation.

In the first stage, the CO₂ absorption column, CO₂ in flue gas is absorbed by a CO₂ lean aqueous ammonia-carbon dioxide solution (NH₃:CO₂ molar ratio >1.5), forming a CO₂ rich solution (NH₃:CO₂ molar ratio ˜1). The remaining gases after the CO₂ is absorbed are released from the absorption column (‘Inert Gases’ in FIG. 1). Similar CO₂ absorption columns are currently employed in the chilled ammonia process.

In the second stage, the solvent mixer, the CO₂ rich ammonia-carbon dioxide solution from the CO₂ absorption column is mixed with an organic solvent (acetone, acetaldehyde, or dimethoxymethane) under mild temperatures and pressures, such as RTP conditions, generating high purity CO₂. The solution becomes CO₂ lean as pure CO₂ is generated.

In the last stage, the solvent distillation column, the solution formed in the solvent mixer enters a distillation column, where the organic solvent is distilled from the CO₂ lean aqueous solution. The aqueous solution is recirculated to the CO₂ absorption column and the organic solvent is recirculated to the solvent mixer.

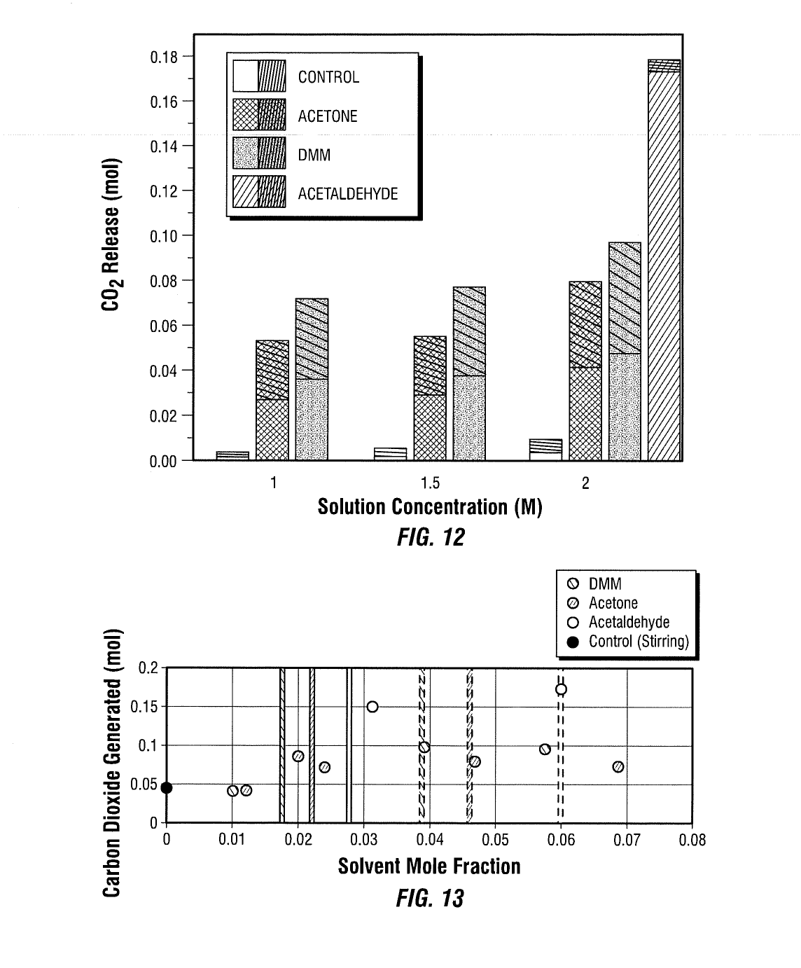

CO₂ Desorption Mechanism: CO₂ was desorbed by adding acetone, dimethoxymethane (DMM), or acetaldehyde to aqueous ammonium bicarbonate solutions under RTP conditions. The graph (FIG. 12) shows the amount of pure CO₂ generated over 1 hour (experimentally observed) and 3 hour (extrapolated) periods when 20 mL of acetone and DMM were added to 100 mL of 1, 1.5, or 2 M aqueous ammonium bicarbonate solutions at RTP conditions. CO₂ desorbed per 20 mL of organic solvent increased with ammonium bicarbonate concentration. Pure CO₂ generation from 2 M ammonium bicarbonate solutions was 51% greater with acetone and 36% greater with DMM than the corresponding results with 1 M ammonium bicarbonate solutions. Furthermore, DMM desorbed greater amounts of pure CO₂ than acetone from 10-30 mL of organic solvent added, despite possessing a lower solvent mole fraction (x_{f}=0.0393 for 20 mL DMM; x_{f}=0.0467 for 20 mL acetone).

FIG. 12 shows: CO₂ generated at different ammonium bicarbonate solution concentrations with different organic solvents injected. Experiments were conducted using an online mass spectrometry setup and 20 mL of solvent added to a 100 mL aqueous ammonium bicarbonate solution. The control was the CO₂(g) desorbed from solution with no organic solvent injected under room-temperature and -pressure (RTP) conditions. Solid bars represent CO₂ generated over 1 h, determined experimentally, and hatched bars represent the additional CO₂ generation during 3 h of operation, from extrapolation. The CO₂ capacity for dimethoxymethane and acetaldehyde added to a 2 M solution is similar to those of current MEA and chilled ammonia processes.

Reports on desalination processes suggest organic solvents precipitate dissolved salts by reducing the dielectric constant (∈ᵣ) of an aqueous solution. Specifically, a reduction in dielectric constant from organic solvent addition weakens the hydration shells surrounding the solvated ions and increases ion association due to the coulombic attraction between oppositely charged ions, thereby triggering salt precipitation. In this study, an organic solvent was added to generate CO₂(g) rather than a solid precipitate. According to studies on the CO₂ absorbing mechanism in aqueous ammonia, aqueous ammonia performs multiple roles as a reactant, catalyst, base, and product controller, thus enabling aqueous phase CO₂ to dissolve at a significantly greater concentration than it would without the presence of ammonia. The addition of an organic solvent may weaken the hydration shells surrounding the dissolved CO₂ due to reduction of the solution dielectric constant, thus prompting the generation of CO₂(g) owing to the significantly lower solubility of aqueous phase CO₂ when its interaction with ammonia is inhibited. DMM's greater CO₂ desorption may be attributed to its significantly lower dielectric constant (DMM ∈ᵣ=2.6; acetone ∈ᵣ=20.7), as DMM requires a lower solvent mole fraction than acetone to decrease the solution dielectric constant by the same magnitude.

Acetaldehyde desorbed more CO₂ than both DMM and acetone, despite having a greater dielectric constant (acetaldehyde ∈ᵣ=21.7) because it possessed a greater solvent mole fraction and a reversible reaction with ammonia species. Acetaldehyde reacts with ammonia under anhydrous conditions to form a trimer. Under aqueous conditions, the acetaldehyde-ammonia trimer is stable at pH above 10, forms the acetaldehyde-ammonia adduct ion at a pH less than 10 and greater than 7, and reversibly dissociates into acetaldehyde and free ammonia at a pH below 7. The aqueous acetaldehyde-ammonia adduct ion, which forms at the pH of aqueous ammonia-carbon dioxide solutions (CO₂ rich pH ˜8; CO₂ lean pH ˜9), decomposes into acetaldehyde vapor and aqueous ammonia upon the volatilization of acetaldehyde. Correspondingly, acetaldehyde desorbed more CO₂ than DMM and acetone and was effectively recovered from the aqueous ammonia-carbon dioxide solution during low temperature distillation.

Negligible CO₂ generation occurs at low solvent concentrations. At 5 mL of organic solvent added to 100 mL of 2 M ammonium bicarbonate, the amount of CO₂ desorbed was 8% less than the no solvent case. This is consistent with a previous study that investigated the use of low concentrations of water soluble organic solvents to prevent the release of ammonia from solution. Specifically, it was found that low concentrations of organic solvents did not influence the rate of CO₂ absorption and desorption.

At high ammonium bicarbonate and solvent concentrations, a plateau in CO₂ generation occurred, as shown in FIG. 13.

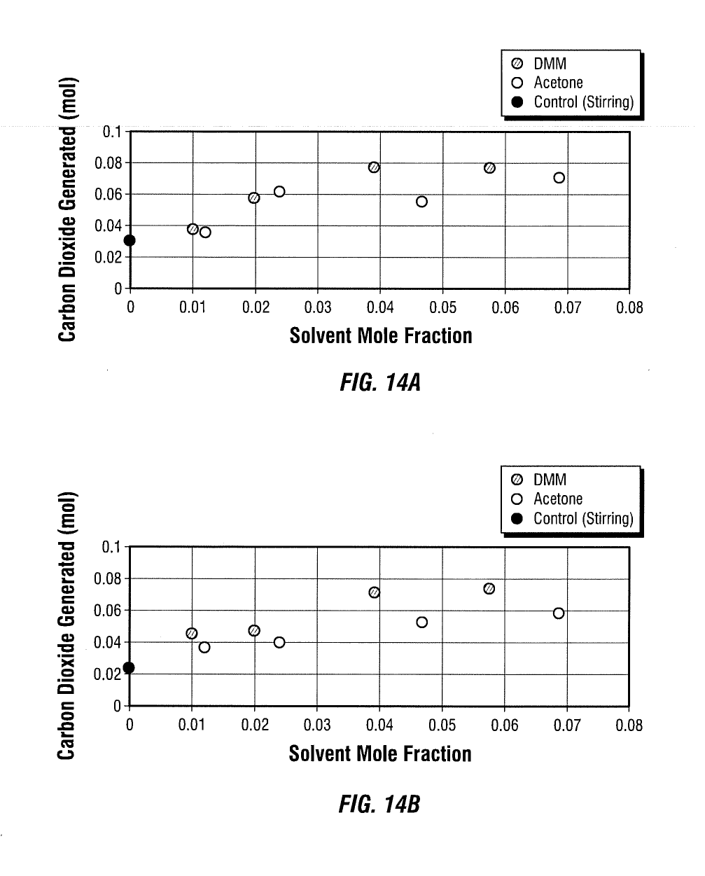

FIG. 14: CO₂ release (moles) as a function of final solvent mole fraction and solvent type for: A) 2 M ammonium bicarbonate, B) 1.5 M ammonium bicarbonate, and C) 1 M ammonium bicarbonate. In (A), solid lines indicate the maximum mole fraction of solvent before eliciting CO₂ gas release (x_{b}=0.0216 for acetone, 0.0181 for dimethoxymethane, and 0.0279 for acetaldehyde). Dashed lines in (A) indicate the optimal organic solvent mole fraction (xf=0.0467 for acetone, 0.0393 for dimethoxymethane, and 0.0599 for acetaldehyde:). Experiments were conducted using the on-line mass-spectroscopy setup with solvent injected into an ammonium bicarbonate solution with stirring at a consistent stir rate.

The CO₂ desorbed from 2 M ammonium bicarbonate solutions at RTP conditions with 30 mL added solvent was 2% and 8% less than 20 mL added solvent for acetone and DMM, respectively, and was accompanied by the immediate formation of solid precipitate. At lower ammonium bicarbonate concentrations (1 M), CO₂ generation increased with higher solvent volumes (20-30 mL) and no precipitate formed. These phenomena may be attributed to an equilibrium between the formation of CO₂ gas and solid precipitate, which shifted toward solid precipitate at higher ammonium bicarbonate and solvent concentrations.

Energy Consumption: The reboiler and condenser heat duties were determined with Aspen HYSYS using the xf and xb organic solvent mole fraction values for each organic solvent in 2 M ammonium bicarbonate. The simulated feed solution contained the xf organic solvent mole fraction and was distilled to form an outlet stream with the xb organic solvent mole fraction. The simulated condenser was cooled using a 20° C. water stream. Acetaldehyde reboiler heat duty was 70% less than DMM and 64% less than acetone due to acetaldehyde possessing a lower boiling point (20.2° C.) and greater xf solvent mole fraction.

The reboiler energy and temperature requirements of this embodiment were compared with present CO₂ capture processes, the chilled ammonia and MEA processes. Present CO₂ capture processes use energy intensive thermal desorption with costly high temperature heat (>120° C.) to generate pure CO₂. This embodiment requires no heat input during CO₂ desorption, instead, desorbing pure CO₂ under room temperature pressure (RTP) conditions through the addition of an organic solvent. The organic solvent is subsequently distilled using abundant low temperature waste heat, resulting in recovery of the solvent and remaining aqueous ammonia-carbon dioxide solution. The energy requirement was calculated by dividing the energy to distill the organic solvent (16.7% v/v) added to 2M aqueous ammonium bicarbonate solution by the mass of pure CO₂ generated.

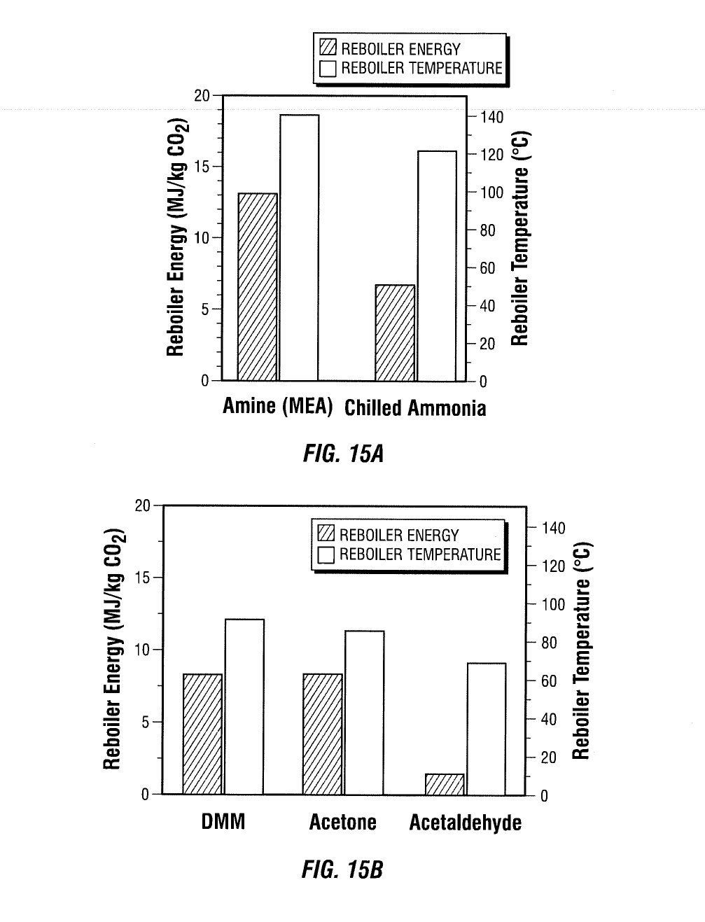

As shown in FIG. 15, the reboiler temperature requirement for acetone and DMM was 49° C. and 55° C. less, respectively, than the MEA process, and 30° C. and 36° C. less, respectively, than the chilled ammonia process. The heat duty for acetaldehyde was 1.39 MJ per kg of CO₂, or less than quarter the heat duty of a pilot chilled ammonia process. The reboiler temperature requirement for acetaldehyde was 68° C., which is 72° C. and 53° C. lower, respectively, than the temperature requirements of the MEA and chilled ammonia processes. Reboiler temperature requirements for all three organic solvents investigated were significantly less than current CO₂ capture technologies and within the temperature range of low grade waste heat.

In this embodiment, aqueous ammonia-carbon dioxide salt decomposition may occur under substantially room temperature and pressure (RTP) conditions using the addition of a water soluble organic solvent to a CO₂ rich aqueous ammonia-carbon dioxide solution (stage 2). Other than mechanical mixing, it may be advantageous for minimal energy to be added during the desorption stage of this embodiment.

Thermal energy is consumed in this during the recovery of the organic solvent from the CO₂ lean aqueous ammonia solution via distillation. Minimal ammonia-carbon dioxide salt decomposition may be intended to occur during this stage. Due to the low temperatures employed and high NH₃:CO₂ molar ratio of the solution, only slight traces of NH₃ vaporize in the distillation column, according to vapor-liquid equilibrium studies. Thus, negligible heat energy may be expended on incidental thermal decomposition of the aqueous ammonia-carbon dioxide salt. Therefore, carbamate decomposition may be negligible in the distillation step.

The energy consumed in the distillation section of this embodiment may be dependent on the relative volatility of the organic solvent to the aqueous solution and the enthalpy of vaporization of the organic solvent. Water soluble solvents with low boiling points, such as acetaldehyde, require significantly lower temperature heat in the distillation and less reflux. Additionally, at these lower temperatures, less water is vaporized, further reducing energy consumption.

Switchable Solvent Addition with Various Recovery Methods

The embodiment uses the addition of a soluble substance or substances to a solution containing CO₂ absorbent—carbon dioxide species, such as ammonia, ammonium, amine, bicarbonate, carbonate, carbon dioxide or carbamate species, to trigger the release of carbon dioxide gas from solution. The added substance recovered from solution via a change in one or more or a combination of system conditions, including, but not limited to, changes in temperature, light, pressure, magnetic field, kinetic energy or a change in the presence of one or more compounds, such as changes in humidity or carbon dioxide concentration. The added substance can be separated and recovered by one or more techniques, including, but not limited to, filtration, centrifuge, decanting, distillation and membrane based process, such as nanofiltration, organic solvent nanofiltration, reverse osmosis, ultrafiltration, membrane distillation, and other membrane based separation devices described herein.

The embodiment may be composed of three main steps: 1) The absorption of CO₂ in a CO₂ lean solution, resulting in the formation of a CO₂ rich solution; 2) The addition of a water-soluble substance or substances to decompose the CO₂ rich solution to a CO₂ lean solution+CO₂(g). This CO₂ gas stream may undergo further purification or treatment to remove water vapor or traces of ammonia or other substances, which may be recycled in the process; 3) The recovery of the added substance or substances using one or more or a combination of changes in system conditions, which may be followed by or integrated with a physical separation mechanism. Changes in system conditions include, but are not limited to changes in temperature, light, pressure, magnetic field, kinetic energy, favorable reaction or a change in the presence of one or more compounds, such changes in the concentration of water vapor or humidity or changes in the presence or headspace concentration of CO₂. The added substance may be physically separated and recovered by one or more techniques, including, but not limited to, filtration, centrifuge, decanting, distillation, magnetism, and/or membrane based process, such as reverse osmosis, forward osmosis, electrodialysis, nanofiltration, organic solvent nanofiltration ultrafiltration, membrane distillation, integrated electric-field nanofiltration, hot nanofiltration, or hot ultrafiltration. The CO₂ lean solution, after the recovery of the added substance or substances may be recycled to the first step of the process. The recovered substance or substances may be recycled to the second step of the process.

### CO₂ Switchable

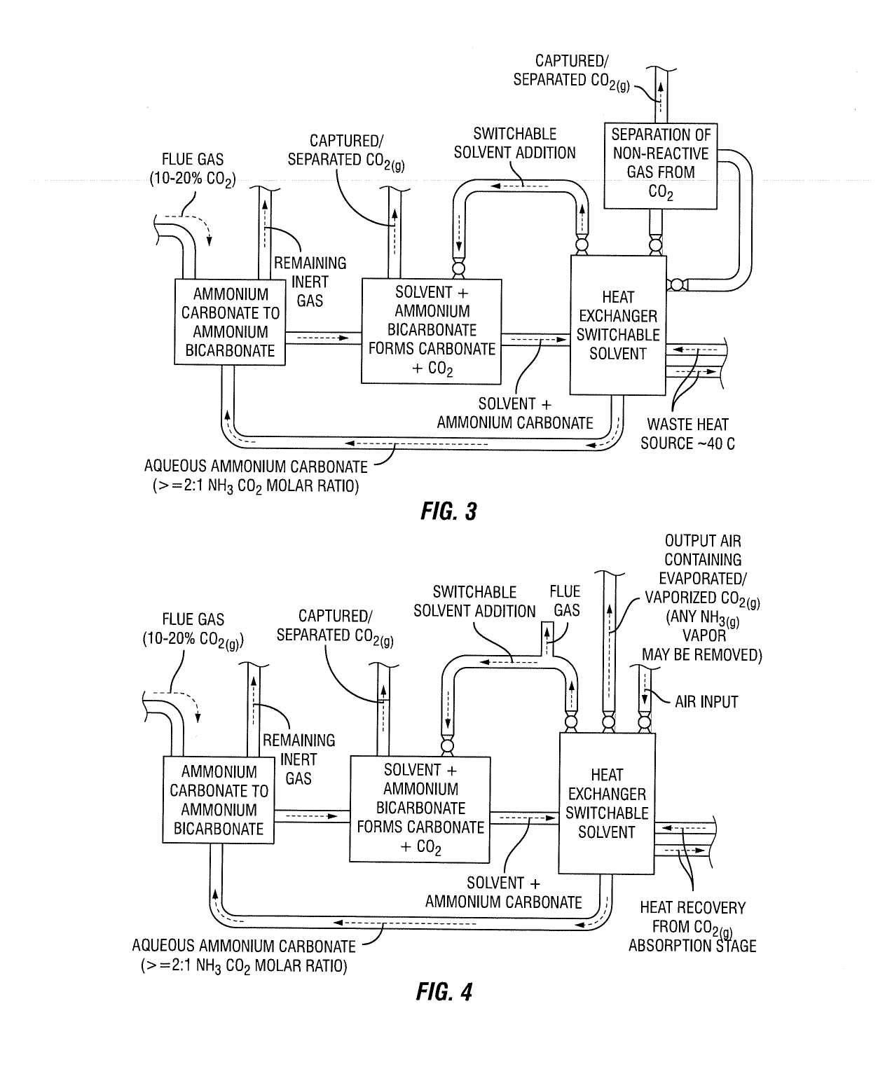

The embodiments described below include those shown in FIG. 3 and FIG. 4.

Description (such as FIG. 3): A switchable solvent may be employed instead of a low boiling point solvent to reduce energy consumption and eliminate the need for a conventional distillation column. Switchable solvents, specifically Switchable Hydrophilicity Solvents (SHS), change their hydrophilicity and solubility through the addition or removal of a substance, usually CO₂, i.e., a CO₂ switchable substance. CO₂ may be added to the switchable solvent to make it hydrophilic. The hydrophilic version of the solvent is then added to the CO₂ rich solution to decompose it into CO₂(g) and CO₂-lean hydrophilic solvent aqueous solution. The switchable solvent is then converted to its hydrophobic form through the application of low grade heat or the use of a non-reactive gas to reduce the partial pressure of CO₂(g) in the headspace and is separated from solution. In the instance where a non-reactive gas is employed, CO₂(g) may be separated from the non-reactive gas through one or more processes, including, but not limited to, the following: gas membrane separation and/or condensation. The non-reactive gas is desired to be insoluble in water, have a much larger molecule size than CO₂ or have a higher boiling point than CO₂. The hydrophobic version of the switchable solvent can be recovered various separation methods described herein including, but not limited to, decanting, centrifuge or membrane.

Description of FIG. 4 or other embodiment with minimal heat input requirement: Switchable solvent without waste heat or recycled inert gas that uses less valuable energy input in recovering the switchable solvent. This embodiment is different from the embodiment shown in FIG. 3 in its process for converting the switchable solvent from its hydrophilic form back to its hydrophobic form. Air is passed through the headspace above the switchable solvent, resulting in the evaporation of CO₂(g) due to the low CO₂(g) partial pressure. As CO₂ in the switchable solvent is desorbed, the solvent switches from its hydrophilic form to its hydrophobic form, forming a two-layer solution. This embodiment doesn't capture the carbon dioxide added to the switchable solvent, which may be absorbed into the switchable solvent in the form of flue gas or other CO₂(g) containing source. However, this embodiment captures large portion of the power plant's CO₂(g), without valuable energy input. A large body of water or ultra-low grade heat source may be applied as a heat source. Additionally, the heat generated during CO₂ absorption may be applied to the switchable solvent regeneration stage, allowing for advantageous cooling of the absorber while supplying heat to the switchable solvent recovery stage. This allows for the only energy input to be the difference in partial pressure between the CO₂(g) in flue gas and in the air. In cases where waste heat is utilized, lower surface area and energy consumption would be required in this system.

### Other Switchable

Overall desirable properties: It has been discovered that there are a wide range of substances capable of being added to an aqueous solution containing a CO₂ absorbent and carbon dioxide to prompt the desorption of gaseous CO₂ desorption. The following is a list of potentially desirable properties for these added substances. Desired properties may include one or more of the following, although the properties are not limited to those described and added substances may or may not exhibit any up to all of these properties.
Reversible solubility in water and/or aqueous solutions
   Miscible or highly or markedly soluble in water or aqueous solutions under certain system conditions
   Immiscible or low solubility or insoluble in water or aqueous solutions under certain system conditions
   Reversible solubility upon changes in system conditions or the presence of stimuli
Low cost recovery from aqueous solution
Low cost substance
Non-hazardous and compatible with most conventional equipment
Does not react with ammonia or carbon dioxide in unfavorable ways
Does not degrade or degrades slowly

Many of these desired properties overlap with those for recoverable forward osmosis draw solutes. Added substance examples and types may include, but are not limited to, recoverable forward osmosis draw solutes, including those described in the review paper http://www.sciencedirect.com/science/article/pii/S2214714414001202, incorporated herein by reference.

### Potential Changes in System Conditions and Example Substances

### Temperature:

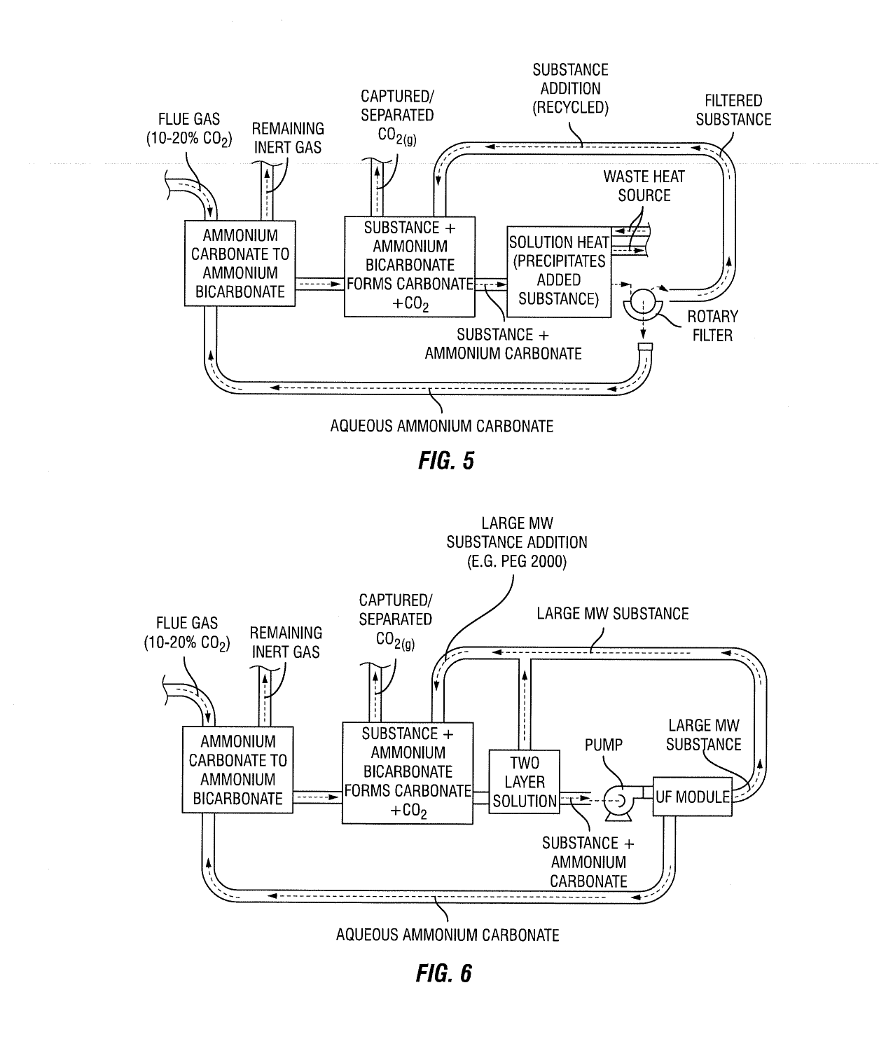

The embodiments described below include those shown in FIG. 5.

Polypropylene glycol 425 is thermally switchable (http://projekter.aau.dk/projekter/files/17652274/Investigation_of_Polypropylene_Glycol_425_a s_a_DrawSolution_for_Forward_Osmosis.pdf which is incorporated herein by reference).

An example of non-toxic, inexpensive thermally switchable substances includes random or sequential copolymers of low molecular weight diols such as 1,2 propanediol, 1,2 ethanediol, and/or 1,3 propanediol. These switchable substances have a cloud point temperature of between 40° C. to 90° C. and a molecular weight high enough to allow for further separation of the substance using nanofiltration. These solutes are used in forward osmosis for desalination. These thermally switchable substances, and other thermally switchable substances, are further described in https://www.google.com/patents/US20120267308, incorporated herein by reference.

Thermally responsive compounds include, but are not limited to, Lower Critical Solution Temperature (LCST) and Upper Critical Solution Temperature (UCST) compounds, thermosensitive magnetic nanoparticles, thermally responsive polyelectrolytes and thermally responsive ionic liquids.

LCST compounds are soluble or have a higher solubility below a certain threshold temperature, the lower critical solution temperature. For example, thermosensitive poly(N-isopropylacrylamide) (PNIPAM) hydrogels can absorb water below the volume phase transition temperature (VPTT, ˜32C) and expel water at temperatures above the VPTT. Other examples of these hydrogel substances include polyacrylamide (PAM), PNIPAM, and poly(Nisopropylacrylamide-co-acrylic acid) and sodium (P(NIPAM-co-SA)). Non-hydrogel LCST compounds include, but are not limited to, Methylcellulose and triethylamine.

Substances may also exhibit a UCST, a temperature which the solution must be above to exhibit more solubility. Many water soluble, non-ionic compounds exhibit both an LCST and a UCST, such as the nicotine-water system.

Examples of thermosensitive magnetic nanoparticles include, but are not limited to those described in the following article http://pubs.rsc.org/en/content/articlelanding/2011/cc/c1cc13944d#!divAbstract which is incorporated herein by reference. These nanoparticles are typically hydrophilic and are coated with various functional groups to allow them to generate osmotic pressure in solution.

Thermally responsive ionic liquids include, but are not limited to those described in the following article http://pubs.rsc.org/en/Content/ArticleLanding/2015/EW/c4ew00073k#!divAbstract which is incorporated herein by reference.

### Light:

Light based solubility change or other form of recovery have been investigated in forward osmosis applications. These include, but are not limited to, P(NIPAM-co-SA) hydrogels with light-absorbing carbon particles.

### Magnetic Field:

Substances showing magnetic field based change in solubility or other form of recovery via changes in magnetic field may be useful. These include, but are not limited to, magnetic nanoparticles with added functional groups (such as those described in http://pubs.acs.org/doi/abs/10.1021/ie100438x, incorporated herein by reference), and magnetic or inductive heating of nanoparticles in solution.

### Pressure:

Substances that change solubility or other recovery method due to pressure or a combination of pressure and temperature may also be useful. These include, but are not limited to, PSA, polyacrylamide (PAM), PNIPAM, and poly(Nisopropylacrylamide-co-acrylic acid sodium (P(NIPAM-co-SA)) hydrogels.

### Kinetic Energy:

Changes in solution kinetic energy can act as a stimulus to change or promote a change in the solubility or other form of recovery of an added substance. Kinetic energy can be of various forms, including, but not limited to, mixing and sonication. Ultrasonic sonication may either increase or decrease solubility and to promote precipitation and crystal nucleation. Ultrasonic sonication may be used to increase the rate of CO₂ desorption.

Mixing may be employed for, including, but not limited to, facilitating the dissolution of the added substance and increase the rate of CO₂ gas desorption.

### Favorable Reaction Embodiments

The general substance embodiment may not involve the added substance chemically reacting with the CO₂ absorbent or CO₂ species. However, if the substance does react with CO₂, the following may be some favorable properties for these reactions:

Properties of a favorable reaction include, but are not limited to one or more or a combination of the following:
Reversibility due to changes in system conditions or stimuli
Reversible binding to ammonia species
Inexpensive reagents
Reversible adduct
Reversible complex ion
Reagents are easily recoverable from solution
Reagents do not degrade or degrade slowly

An example of a potentially favorable reversible reaction includes the formation of reversible ammonia-metal complexes. These complexes may reduce the affinity of ammonia to the carbon dioxide in solution, resulting in a release or a low temperature release of carbon dioxide from solution.

In some favorable reactions, the reagents may function as catalysts. Instances may also exist where the favorable reaction does not involve reversibility. In the instance where the reaction is irreversible, it may be advantageous for one or more byproducts to have a value-added application, such as use in forward osmosis desalination or fertilization.

Preventing Unfavorable Reaction with Ammonia or Carbon Dioxide Species:

Some substances with favorable properties, may react or interact with ammonia and or carbon dioxide species in potentially unfavorable ways within the process unless additional measures are taken. Substances that have a greater likelihood of reaction with ammonia or ammonium salts include those with Amine, Amide, Ketone, Aldehyde, Ester, or Carboxylic Acid functional groups. If substances on their own react with the ammonia or carbon dioxide species in potentially unfavorable ways, it may be advantageous for the reaction to be slow or the reaction may be inhibited with the addition of another substance or a change in system conditions.

### Alternative Embodiments

Embodiment with at Least Partial Thermal Decomposition

The addition or presence of a water-soluble substance, such as an organic solvent, to a CO₂ rich aqueous ammonia-carbon dioxide solution may generally initiate and foster CO₂ desorption independently of temperature. However, heat, including heat above the decomposition temperature of the absorbent—carbon dioxide, may be applied to the CO₂ desorption and substance regeneration stages. This may, include, but not be limited to, increase CO₂ desorption rate, increase solution capacity, reduce CO₂ loading, improve the properties of the CO₂ absorption solution to maximize CO₂ uptake, improve the properties of the CO₂ absorption solution to maximize the rate of CO₂ uptake, help overcome enthalpy of desorption or overcome activation energy, which may be especially useful for CO₂ absorbents with high enthalpies of reaction relative to ammonia. The presence of the soluble substance, such as PEG or PPG, may reduce the temperature and energy requirements of CO₂ desorption during thermal desorption in comparison to existing ammonia or amine thermal desorption processes.

### Example 1

A water soluble substance is added at a moderate or cool temperature, such as at room temperature, in the CO₂ desorption stage and gaseous CO₂ is desorbed. After at least a significant portion, such as less than any of the following: 5%, or 10%, or 15%, or 20%, or 25%, or 30%, or 35%, or 40%, or 45%, or 50% of the CO₂ in solution, is desorbed, heat may be applied to the mixed desorption solution in a separate or the same reactor or reactors. It may also be desirable for heat to be applied when the CO₂ desorption rate due to the presence of the soluble substance has appreciably subsided, such as the CO₂ desorption rate subsiding to less than any of the following: 95%, or 90%, or 75%, or 60%, or 50% or 40%, or 30%, or 20%, or 10% of the maximum CO₂ desorption rate after soluble substance injection. The application of heat may enhance CO₂(g) desorption. The temperature and energy requirements for thermal desorption may be significantly less than conventional thermal desorption processes due to the presence of the water-soluble substance. It may be desirable for the added substance in this example to be non-volatile, such as a vapor pressure at 20° C. at less than 0.1 atm, or any of the following: 0.05 atm, or 0.03 atm, or 0.01 atm, or 0.001 atm, or 0.0001 atm, and minimally prone to thermal degradation. Such substances, include, but are not limited to, PEG and PPG. This embodiment may allow for significant reductions in energy requirements for CO₂ capture, while allowing for, including, but not limited to, one or more or a combination of the following: greater CO₂ desorption rates, greater CO₂ solution capacity, lower precipitate formation, lower CO₂ loading in the CO₂ lean solution, and greater CO₂ uptake in the CO₂ absorption column.

### Example 2

The water-soluble substance is added to the CO₂-rich solution in the CO₂ desorption stage and heat is applied to the solution during most the CO₂ desorption timeframe. Depending on the concentration of the added substance, the rate and temperature of heat input, residence time, the rate of mixing and other factors, the initial CO₂ desorption may be primarily due to the influence of the soluble substance. During this initial timeframe, the heat may, include, but not be limited to, increase the rate of CO₂ desorption or facilitate CO₂ desorption. Depending on many factors, the influence of heat application on CO₂ desorption may increase over time and the influence of the added substance may subside. It may be desirable for the added substance in this example to be relatively non-volatile and not prone to substantial thermal degradation. Such substances, include, but are not limited to, PEGs and PPGs.

### Example 3

In embodiments where the added substance is recovered using heat, such as embodiments with various forms of distillation or switchable solvent, or where heat is applied during soluble substance recovery, such as may be the case in membrane-based recovery embodiments, CO₂(g) may be desorbed. This CO₂ desorption may be in part due to thermal decomposition. This CO₂ may be recovered and utilized in a similar manner to the CO₂ desorption during the CO₂ desorption stage.

Soluble Substance Addition Carbon Capture with ‘Salting-Out’ Solvent Recovery

The embodiments described below include those shown in FIG. 6.

In this embodiment, following soluble substance addition, CO₂(g) is generated until the NH₃:CO₂ molar ratio in the aqueous solution is sufficient to prompt ‘salting-out’ or the formation of a multi-layer solution. This NH₃:CO₂ molar ratio may be greater than 1.5:1. The presence of aqueous ammonium carbamate species may facilitate the formation of a two layer solution. After the formation of the two or more-layer solution, the layer with a lower concentration of large molecular weight solvent is fed into a separation mechanism, which includes those described in FIG. 1. The layer with a higher concentration of solvent may also be fed into one or more of these separation mechanisms if desired. In the suggested process, the layer with a higher concentration of solvent is combined with the concentrate formed during the separation mechanism, forming a high concentration solvent solution. In this instance, the process requires less energy due to separating solvent from a smaller volume of solution. The layers may be separated via various processes, including, but not limited to, decanting or centrifugation. In another instance, no separation mechanism is employed after the two or more layers are separated. In this instance, it may be advantageous for a relatively high concentration of soluble substance to be present in one or more layers and relatively low concentration of soluble substance to be present in one or more layers. The layer or layers with a lower concentration of the soluble substance may be transferred to the absorption column as the absorption solution. The layers with a higher concentration of the soluble substance are transferred to the CO₂ desorption step. In the instance where lower molecular weight or boiling point solvents are used, a distillation process may be used to recover the solvent in one or more of the solvent layers. In the instance where the added substances are capable of being separated via a membrane, including, but not limited to, high molecular weight substances, a membrane may be used to concentrate or recover the substance or purify the CO₂ lean or CO₂-rich solutions in one or of the substance containing layers.

### Ultra-Low Boiling Point Water Soluble Solvent Addition Carbon Capture System

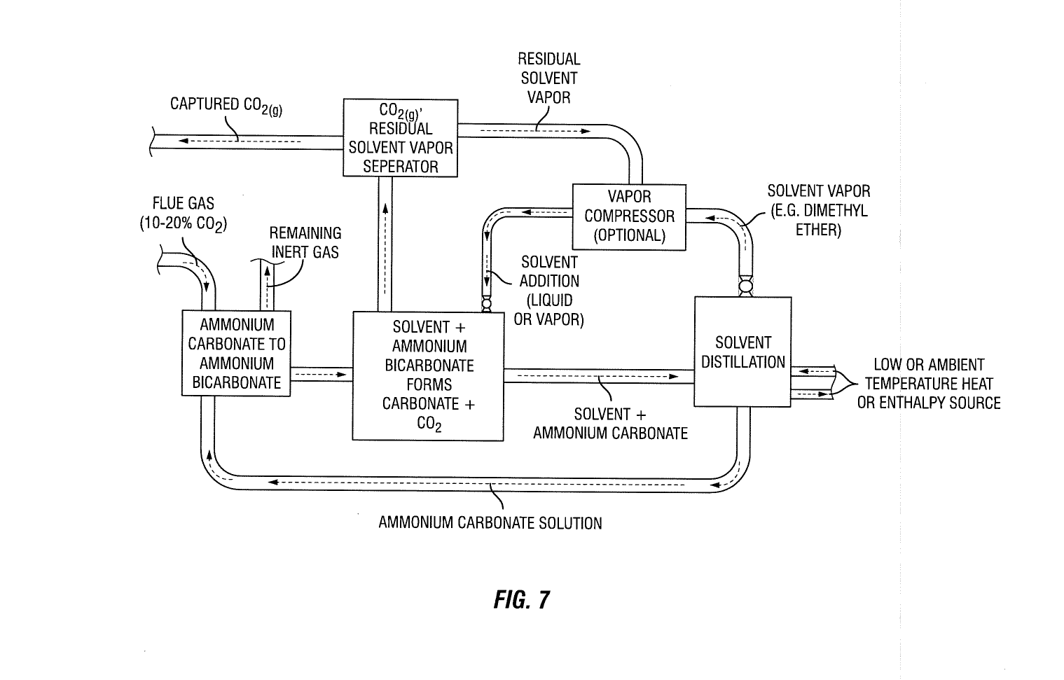

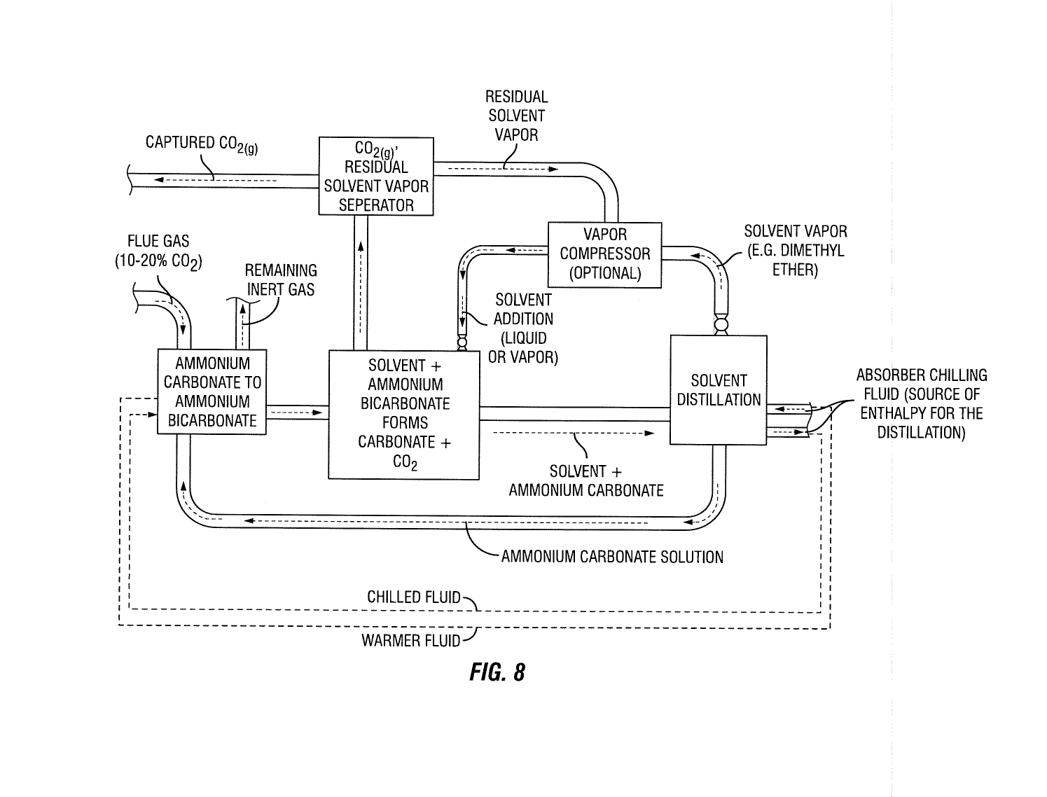

The embodiments described below include those shown in FIG. 7 and FIG. 8.

This embodiment is composed of three main steps: 1) The contacting of a gas containing CO₂ to convert a CO₂ lean solution to a CO₂ rich solution. The remaining inert gases may undergo further purification, treatment or compression; 2) The addition of low boiling point soluble substance or substances, such as dimethyl ether, to the CO₂ rich solution to generate CO₂(g), creating a CO₂ lean solution+added substance+CO₂(g). The substance may be added in the gas phase, liquid phase or a combination of gas and liquid phases. This CO₂(g) stream may undergo further purification, treatment or compression. Any remaining or residual solvent vapor in the CO₂(g) stream may be separated and recovered; 3) The recovery of the added substance or substances using ultra-low temperature distillation. Heat or enthalpy sources include, but are not limited to, ultra-low temperature waste heat sources, ambient temperature enthalpy sources, and chilling fluids. The process may replace or greatly minimize the need for evaporative cooling towers, as the distillation column can cool the condenser fluids in an open or closed loop. Higher temperature heat may be used if desired. The process may be conducted without a vapor compressor and may condense the solvent with lower temperatures or only condense a portion of the pure solvent to the liquid phase before solvent addition. The process may be conducted under a higher pressure, allowing for the solvent to condense under more moderate conditions without a compressor. With a vapor compressor or mechanical vapor compression distillation, the solvent may condense at a greater temperature and residual solvent vapors may be easier to recover. Heat may be recovered or removed during to solvent vapor condensation or compression. The CO₂(g) may need to be separated from the low boiling point solvent vapor during CO₂ desorption. This may involve various treatment methods, including, but not limited, water wash-down, condenser, compression or other systems and methods described herein.

In another embodiment, FIG. 8, the enthalpy or heat source is a fluid exchanged with the CO₂(g) absorption column. The fluid or chilling fluid may be an external fluid heat exchanged with the absorption and distillation columns or the solutions within either or both the absorption or distillation columns. Additional heat or enthalpy may be recovered from the residual vapor separator and the vapor compressor. CO₂(g) absorption is known in the art to perform more advantageously at ambient or lower than ambient temperatures. Typically, in, for example, the chilled ammonia process, an external refrigeration, chilling or evaporative cooling unit is used to cool the solution, increasing energy load and capital and operator costs. This chilling is generally required due to the exothermic nature of CO₂(g) absorption reaction. The embodiment shown in FIG. 8 allows for the heat energy generated in CO₂(g) absorption to be recovered or used to power the solvent distillation. Additionally, heat or enthalpy sources may be used, however, it may be advantageous to integrate and balance the energy demands in the process, including those from the CO₂(g) absorption and solution regeneration stages.

Generally desirable ultra-low boiling point substances: Desired substances may include one or more of the following, although the properties are not limited to those described and added substances may or may not exhibit any of these properties.
High solubility in water and/or aqueous solutions
Low cost
Low boiling point
Non-hazardous and compatible with most conventional equipment
Does not react with ammonia or carbon dioxide in unfavorable ways
Does not degrade or degrades slowly or degradation can be inhibited

An example solvent that meets this criteria is dimethyl ether. Dimethyl ether exhibits high solubility in water, even above its boiling point, is essentially non-toxic and is a low cost, commodity chemical. Dimethyl Ether may be sufficiently soluble in water for this application under moderate conditions (see graph below). Based on its molar mass and dielectric constant, the process may require a mole fraction of 0.04-0.06 to prompt CO₂ desorption, which may be achieved under moderate conditions (http://www.pet.hw.ac.uk/icgh7/papers/icgh2011Final00008.pdf, incorporated herein by reference).

In another embodiment, the solvent distillation is used for chilling an external medium. This may include, but is not limited to, cooling condenser fluid from power generation, HVAC systems, ice skating rinks, datacenters, manufacturing, industrial processes, solar thermal or photovoltaic and mining and natural resource extraction. Natural heat sinks may also be used as enthalpy or heat sources including, but not limited to, water bodies, air, geothermal sources, and solar thermal sources.

### Adsorbent Embodiment

Substance is added to a CO₂ adsorbent, such as quaternary ammonium cation containing material, to desorb CO₂.

In the case of adsorbents, the adsorbents may exhibit any range of surface areas or surface morphologies.
CO₂ capture adsorbents and hybrid adsorbents—absorbents may exhibit properties, including, but not limited to, one or more or a combination of the following:
   Desorption of CO₂ at least partially due to contact with an added substance
   Insoluble or exhibits low solubility in the added substance
   Exhibits high solubility in the added substance
   Exhibits high solubility in the added substance when not bonded with CO₂
   Exhibits high solubility in the added substance at higher Adsorbent:CO₂ molar ratios
   Dissolves in the solvent water solution while releasing CO₂(g)
   Exhibits high solubility in the added substance when bonded with CO2
   Exhibits high solubility in the added substance at lower Adsorbent:CO₂ molar ratios
   Exhibits high solubility in a solution media with a substance
   Exhibits low solubility in a solution media with a substance
   Changes in surface wetting due to contact with a substance
   Changes in surface morphology due to contact with a substance
   Decrease in activation energy due to presence of a substance
   Reduction in CO₂ desorption energy requirement

General Conditions and System Technicalities Regarding Systems and Methods of the Integrated Process:
Distilled solvent or solvent vapor may be contacted with the second stage of the embodiment shown in FIG. 2, as a means of added the solvent to desorb CO₂(g). The vapor may dissolve and condense, adding solvent to the solution, while increasing the solution temperature, which may improve CO₂(g) desorption yield and recover heat or a portion of the enthalpy of vaporization. Additionally, any CO₂(g) released from solution during the distillation is combined with the CO₂(g) released during the second stage. This may reduce energy consumption in preheating the solution prior to distillation and lower capital costs by minimizing or eliminating the need for a condenser.
Ammonium Carbonate may have an ammonia to carbon dioxide molar ratio of >1.5:1 to <100:1
Ammonium Bicarbonate or Ammonium Sesquicarbonate may have an ammonia to carbon dioxide molar ratio of 0.25:1 to 1.5:1
Solvent or substance may be substance or combination of substances that when added to a carbon dioxide species containing solution, such as an ammonia-carbon dioxide solution, prompts the release of carbon dioxide. The solvent or substance may include, but is not limited to, one or more of the following: a soluble substance, a water soluble substance, an organic solvent, an organic substance, a soluble organic substance, a water soluble organic solvent, a soluble polymer, a water soluble organic substance, a substance containing carbon, a substance containing carbon and hydrogen, a substance containing carbon, hydrogen and oxygen, or a substance containing hydrogen and nitrogen, a non-ionic substance, a non-reactive substance, a non-ionic water soluble substance, non-reactive water soluble substance, inert soluble substance, inert water soluble substance, or inert substance.
Switchable Solvent: Include substances with Switchable Hydrophilicity (SHS), Switchable Polarity (SPS), Switchable Water (SW). Further information is incorporated herein by reference: http://pubs.rsc.org/en/Content/ArticleLanding/2012/EE/c2ee02912j#!divAbstract
Higher temperature heat may be utilized
Catalysts: Substance(s) may be added or included at any component in the system to enhance performance. These improvements in performance may include, but are not limited to, enhancing CO₂ absorption, enhancing CO₂ desorption, prevention of ammonia reaction with solvent and preventing ammonia slip. Examples of absorption and desorption catalysts known in the art, include, but are not limited to, HZSM-5, γ-Al₂O₃, HY, silica-alumina, or combinations thereof
Waste Heat/Low Grade Heat: Heat energy that can be utilized in the systems and methods described herein. The temperature me be less than 200° C., or less than 100° C., or less than 50° C. It may be advantageous for the heat source to be an untapped byproduct of another process. Examples of waste heat sources include, but are not limited to, the following: Power Plant (Natural gas, coal, oil, petcoke, biofuel, municipal waste), Condensing water, Flue Gas, Steam, Oil refineries, Metal production/refining (Iron, Steel, Aluminum, etc.), Glass production, Manufacturing facilities, Fertilizer production, Transportation vehicles (ships, boats, cars, buses, trains, trucks, airplanes), Waste Water Treatment, Solar thermal, Solar pond, Solar photovoltaic, Geothermal (Deep Well), Biofuel powered vehicles, Biofuel/Biomass/Municipal Waste Power Plants, Desulfurization, Alcohol production, hydrogen sulfide treatment, acid (e.g. sulfuric) production, Renewable fertilizer production, Ocean Thermal, Space heating, Grey water, Diurnal temperature variation, Geothermal (Shallow well/loop), or respiration.
Carbon Dioxide Sources: Any process or resource producing or containing carbon dioxide. Examples of CO₂ sources include, but are not limited to, the following: Power Plant (Natural gas, coal, oil, petcoke, biofuel, municipal waste), Waste Water Treatment, Landfill gas, Air, Metal production/refining (Iron, Steel, Aluminum, etc.), Glass production, Oil refineries, HVAC, Transportation vehicles (ships, boats, cars, buses, trains, trucks, airplanes), Natural Gas, Biogas, Alcohol fermentation, Volcanic Activity, Decomposing leaves/biomass, Septic tank, Respiration, Manufacturing facilities, Fertilizer production, Geothermal processes where CO_{2(g)} releases from a well or wells.
Heat or cooling may be applied at any point in the process. For example, heat may be applied in the substance addition and mixing stage (Stage 2) for various purposes, including, but not limited to, promoting CO₂(g) generation and increasing mixing rate and cooling may be applied in the absorption column.
Heat exchangers and recovery devices may be employed where advantageous. For example, heat may be recovered from the streams exiting the distillation column by preheating the solution entering the distillation column.
All Embodiments: The gases considered “inert” may not react with the ammonia or carbon dioxide in unfavorable ways. These gases may not be universally “inert,” as they may react with other substances or under other or similar conditions. These “inert gases” may include, but are not limited to, nitrogen, oxygen, hydrogen, argon, methane, carbon monoxide, low concentrations of CO₂(g) volatile hydrocarbons, such as ethane, butane, propane. The “flue gas” or carbon dioxide containing gas stream may include any gas stream that at least partially comprises carbon dioxide.
Degradation, oxidation and corrosion prevention
   Absorbent Degradation or Oxidation: Degradation or oxidation of the CO₂ absorbent may occur due to, including, but not limited to, one or more or a combination of the following: thermal degradation, light, UV light, or reaction with oxygen, NOₓ, SOₓ, CO₂ or the added substance. Degradation or oxidation is known in the art to be most prevalent in amine and azine CO₂ absorbents. Degradation or Oxidation inhibitors include, but are not limited to, one or more or a combination of the following: antioxidants, sulfites, bisulfite, metabisulfites, nitrites, hydroxyethylidene diphosphonic acid (HEDP), diethylene triamine penta acetic acid (DPTA), diethylenetriamine penta (methylene phosphonic acid) (DTPMP), ethylenediamine tetra (methylene phosphonic acid) (EDTMP), citric acid, or absorbent combinations that inhibit degradation or oxidation.
   Added Substance Degradation or Oxidation: The added substance may exhibit oxidation or degradation. Measures may be employed to prevent degradation and oxidation. Degradation or oxidation of the added substance may occur due to, including, but not limited to, one or more or a combination of the following: thermal degradation, light, UV light, or reaction with oxygen, NOₓ, SOₓ, CO₂, or the CO₂ absorbent. Degradation or Oxidation inhibitors include, but are not limited to, one or more or a combination of the following: antioxidants, sulfites, bisulfite, metabisulfites, nitrites, or added substance combinations that inhibit unfavorable reactions, such as degradation or oxidation.
   Vessels and Equipment Corrosion: Vessels and equipment at least partially resilient to degradation and corrosion in the presence of the reagents employed in the integrated process may be implemented. Corrosion resistant materials may include, but is not limited to, one or more or a combination of the following: Teflon, polyethylene, polypropylene, PVC, stainless-steel, metals non-reactive with ammonia, metals non-reactive with aqueous ammonium, and materials not reactive with the CO₂ absorbent or absorbents employed.
Mixing devices, include, but are not limited to, on or more or a combination of the following:
   CSTR, Batch, Semibatch, or flash devices
   Turbine
      Rushton Turbine
      Smith Turbine
      Helical Turbine
      Bakker Turbine
   Low shear mixer, High shear mixer, Dynamic mixer, Inline mixer, Static mixer, Turbulent flow mixer, No mixer, Close-clearance mixer, High shear disperser, Static mixers, Liquid whistles, Mix-Itometer, Impeller mixer, Liquid-Liquid mixing, Liquid-Solid mixing, Liquid-Gas mixing, Liquid-Gas-Solid mixing, Multiphase mixing, Radial Flow, Axial Flow, Flat or curved blade geometry
Any portion of the process may be heated or cooled. Heat sources may include, but are not limited to, waste heat, power plant waste heat, steam, heat, pump or compressor waste heat, industrial process waste heat, steel waste heat, metal refining and production waste heat, paper mill waste heat, factory waste heat, petroleum refining waste heat, solar heat, solar pond, air conditioner waste heat, combustion heat, geothermal heat, ocean or water body thermal heat, stored heat, and CO₂(g) absorption solution heat.
The solution may comprise one or more or a combination of the following phases throughout the integrated process: liquid, solid, liquid-solid slurry, liquid-solid mixture, gas, two-phase solution, three-phase solution, two-layer solution, or supercritical
The CO₂ rich compound or CO₂ may be captured or absorbed prior to the integrated process. In this instance, the CO₂ desorption stage may be directly fed a CO₂ rich solution by a device or stage other than an absorption column. The CO₂ may have been absorbed in a separate location and the resulting CO₂ rich feed is transported to the CO₂ desorption stage.
   The CO₂ in the CO₂ rich compound may not have been captured from a gas source. The CO₂ instead may be sourced from a solid or liquid, which may be directly fed into the process or undergo methathesis or displacement reaction to remove extract this CO₂ species into a form with which CO₂ can be desorbed with substance addition. An example of this may include CO₂ species derived from a metathesis reaction with limestone or a metathesis reaction in the production of another CO₂ containing compound. Another example may be CO₂ species present in compounds in waste water, such as Urea or ammonium carbonate or ammonium bicarbonate.

Soluble Substances Lists:

Water soluble substances may include, but are not limited to, the substances detailed below:

Overview of substances: Aqueous solution, Water soluble polymer, Soluble polymer, Glycol Polyethylene Glycol, Polypropylene Glycol Ethers, Glycol Ethers, Glycol ether esters, Triglyme. Polyethylene Glycols of multiple geometries, Methoxypolyethylene Glycol, Polyvinyl Alcohol Polyvinylpyrrolidone, Polyacrylic Acid, Diol polymers, 1,2 propanediol, 1,2 ethanediol, 1,3 propanediol, Cellulose Ethers, Methylcellulose, Cellosize, Carboxymethylcellulose, Hydroxyethylcellulose, Sugar Alcohol, Sugars, Alcohols Ketones, Aldehydes, Esters, Organosilicon compounds, Halogenated solvents

### Non-Volatile Substances:

Poly(ethylene glycol) (PEG) and Poly(ethylene oxide) (PEO)
   Heterobifunctional PEGs
      Azide (—N3) Functionalized
      Biotin Functionalized
      Maleimide Functionalized
      NHS Ester Functionalized
      Thiol Functionalized
      COOH Functionalized
      Amine Functionalized
      Hydroxyl Functionalized
      Acrylate/Methacrylate Functionalized
   Homobifunctional PEGs
   Monofunctional PEGs
   PEG Dendrimers and Multi-arm PEGs
      PEG-core Dendrimers
      Multi-arm PEGs
      Multi-arm PEG Block Copolymers
   PEG Copolymers
      PEG Diblock Copolymers
      PEG/PPG Triblock Copolymers
      Biodegradable PEG Triblock Copolymers
      Multi-arm PEG Block Copolymers
      Random Copolymers
   PEG and Oligo Ethylene Glycol
      Examples: PEG 200, PEG 300, PEG 400, PEG 600, PEG 1000, PEG 1450, PEG 1500, PEG 2050, PEG 3350, PEG 8000, PEG 10000
   Poly(ethylene oxide)
   High Oligomer Purity PEG
   Polyethylene glycol-polyvinyl alcohol (PEG-PVA)
Polypropylene Glycol (PPG)
   Examples: PPG 425-4000
Poly(N-isopropylacrylamide) (PNIPAM) and Polyacrylamide (PAM)
   PNIPAM Copolymers
   Poly(N-isopropylacrylamide) (PNIPAM)
   Polyacrylamide (PAM) and Copolymers
Poly(2-oxazoline) and Polyethylenimine (PEI)
Poly(acrylic acid), Polymethacrylate and Other Acrylic Polymers
Poly(vinyl alcohol) (PVA) and Copolymers
   Poly(vinyl alcohol) (PVA)
   Poly(vinyl alcohol-co-ethylene) ethylene
Poly(vinylpyrrolidone) (PVP) and Copolymers
Polyelectrolytes
   Poly(styrenesulfonate) (PSS) and Copolymers
   Polyacrylamide (PAM)-based Polyelectrolytes
   Poly(acrylic acid) (PAA), Sodium Salt
   Poly(allylamine hydrochloride)
   Poly(diallyldimethylammonium chloride) Solution
   Poly(vinyl acid)
   Miscellaneous-(1)
Cucurbit[n]uril Hydrate
Quaternary ammonium polymers
Carboxypolymethylene (carbomer)
Polyvinyl methyl ether-maleic anhydride (PVM-MA)
Carboxypolymethylene (carboxyvinyl polymer)
Polyvinyl methyl ether-maleic anhydride
Carboxymethylcellulose
Hydroxyethylcellulose and derivatives
Methylcellulose and derivatives
Other cellulose ethers
   Ethylcellulose
   Hydroxypropylcellulose
Sodium carboxymethylcellulose
Hydroxyethylcellulose and ethyl hydroxyethylcellulose
Natural water-soluble polymers: Starches, Sugars, Polysaccharides, Agar, Alginates, Carrageenan, Furcellaran, Casein and caseinates, Gelatin, Guar gum and derivatives, Gum arabic, Locust bean gum, Pectin, Cassia gum, Fenugreek gum, Psyllium seed gum, Tamarind gum, Tara gum, Gum ghatti, Gum karaya, Gum tragacanth, Xanthan gum, Curdlan, Diutan gum, Gellan gum, Pullulan, Scleroglucan (sclerotium gum)
   PEGs are available with different geometries, including, but not limited to, the following:
Branched PEGs: have three to ten PEG chains emanating from a central core group.
Star PEGs: have 10 to 100 PEG chains emanating from a central core group.
Comb PEGs: have multiple PEG chains normally grafted onto a polymer backbone.

Substance Details for embodiments, including, but not limited to, one or more or a combination of the following:
Soluble substance
Soluble organic solvent
Soluble polymer
Water soluble substance
Soluble substance separable with a membrane
Water soluble substance separable with a membrane
Water soluble organic solvent
Water soluble polymer
Organic solvent separable with a membrane
Polymer separable with a membrane
Soluble organic solvent separable with a membrane
Soluble polymer separable with a membrane
Large molecular weight water soluble organic solvent
Small molecular weight water soluble polymer
Non-volatile organic solvent
Low volatility organic solvent
High volatility organic solvent that is separable with a membrane
Organic solvent with a molecular weight, including, but not limited to, greater than 100 da or any of the following: 125 da, or 150 da, or 175 da, or 200 da, or 225 da, or 250 da, or 275 da, or 300 da, or 325 da, or 350 da, or 375 da, or 400 da, or 425 da, or 450 da, or 475 da, or 500 da, or 525 da, or 550 da, or 575 da, or 600 da
Polymer with a molecular weight, including, but not limited to, greater than 100 da or greater than any of the following: 125 da, or 150 da, or 175 da, or 200 da, or 225 da, or 250 da, or 275 da, or 300 da, or 325 da, or 350 da, or 375 da, or 400 da, or 425 da, or 450 da, or 475 da, or 500 da, or 525 da, or 550 da, or 575 da, or 600 da
Substance with a molecular weight, including, but not limited to, greater than 100 da or greater than any of the following: 125 da, or 150 da, or 175 da, or 200 da, or 225 da, or 250 da, or 275 da, or 300 da, or 325 da, or 350 da, or 375 da, or 400 da, or 425 da, or 450 da, or 475 da, or 500 da, or 525 da, or 550 da, or 575 da, or 600 da
Organic solvent with a hydration radius, including, but not limited to, greater than 100 da, or greater than any of the following: 125 da, or 150 da, or 175 da, or 200 da, or 225 da, or 250 da, or 275 da, or 300 da, or 325 da, or 350 da, or 375 da, or 400 da, or 425 da, or 450 da, or 475 da, or 500 da, or 525 da, or 550 da, or 575 da, or 600 da
Polymer with a hydration radius, including, but not limited to, greater than 100 da, or or greater than any of the following: 125 da, or 150 da, or 175 da, or 200 da, or 225 da, or 250 da, or 275 da, or 300 da, or 325 da, or 350 da, or 375 da, or 400 da, or 425 da, or 450 da, or 475 da, or 500 da, or 525 da, or 550 da, or 575 da, or 600 da
Substance with a hydration radius, including, but not limited to, greater than 100 da, or or greater than any of the following: 125 da, or 150 da, or 175 da, or 200 da, or 225 da, or 250 da, or 275 da, or 300 da, or 325 da, or 350 da, or 375 da, or 400 da, or 425 da, or 450 da, or 475 da, or 500 da, or 525 da, or 550 da, or 575 da, or 600 da

Example Solvents Capable of Being Rejected By Ultrafiltration
or Relatively Larger Pore Size Nanofiltration

| | Melting | | Azeotrope w/ | | | Molar | |
|---|---|---|---|---|---|---|---|
| Solvent | Point | Dielectric | Water | Azeotrope | | Mass | |
| Name | (° C.) | Constant | (Yes/No) | Composition | Solubility | (g) | Toxicity |
| PEG-2000 | | | | | | | |
| PEG-1000 | 35-40 | | | | 80 | 1000 | |
| PEG-1450 | 42-46 | | | | 72 | 1450 | |
| PEG-3350 | 53-57 | | | | 67 | 3350 | |
| PEG-4000 | 53-59 | | | | 66 | 4000 | |
| PEG-4600 | 54-60 | | | | 65 | 4600 | |
| PEG-8000 | 55-62 | | | | 63 | 8000 | |
| PEG-10000 | | | | | | | |
| PEG-12000 | | | | | | | |
| PEG-20000 | | | | | | | |
| PEG-35000 | | | | | | | |
| PVA (9000- | | | | | | | |
| 10000) | | | | | | | |
| PVA (13000- | | | | | | | |
| 23000) | | | | | | | |

Rejected By: Nanofiltration

| | | | | | | | | |
|---|---|---|---|---|---|---|---|---|
| | Melting | | Azeotrope | | | Molar | Reacts | |
| Solvent | Point | Dielectric | w/Water | Azeotrope | Solubility | Mass | with | |
| Name | (° C.) | Constant | (Yes/No) | Composition | in Water | (g) | NH3? | Toxicity |

| | | | | |
|---|---|---|---|---|
| PEG-400 | 4-8 | Completely | 400 | No |
| PPG-425 | | Completely | 425 | |
| PEG-600 | 15-25 | Completely | 600 | No |
| Methoxy- | −5-10 | Completely | 350 | No |
| polyethylene | | | | |
| Glycol-350 | | | | |
| Sucrose | Decomposes | Completely | 342.30 | Yes |
| Methoxy- | 15-25 | Completely | 550 | No |
| polyethylene | | | | |
| Glycol-550 | | | | |
| Methoxy- | 27-32 | Completely | 750 | No |
| polyethylene | | | | |
| Glycol-750 | | | | |
| Tannic Acid | | | 1701 | Probably |

### Volatile Substances:

Ultra-Low Temperature Boiling Point Solvents and Azeotropes

| | | | Azeotrope | | | Ethalpy | | |
|---|---|---|---|---|---|---|---|---|
| | Boiling | Dielectric | H₂O | Azeotrope | | of Vap | MW | |
| Solvent Name | Point | Constant | (Yes/No) | Composition | Solubility | (kJ per mol) | (g) | Toxicity |
| Dimethyl- | −24 C. | | No | | 34% (20 C.) | | 46.07 | 1, Fire |
| ether | | | | | | | | Hazard |
| Ethyl Methyl | 7.4 C. | | No | | Soluble | | 60.1 | 1, Fire |
| Ether | | | | | | | | Hazard |
| Acetal- | 20 C. | | No | 0.8437 | | | | |
| dehyde- | | | | (Acetaldehyde) | | | | |
| Diethyl Ether | | | | | | | | |

Low Boiling Point Solvents List

| | | | | | | | Ethalpy | |
|---|---|---|---|---|---|---|---|---|
| | Boiling | | Dielectric | Azeotrope | | | of Vap | |
| Solvent Name | Point | | Constant | (Yes/No) | pKb | Solubility | (kJ per mol) | Toxicity |
| Methyl Formate | 32 | C. | 8.5 | No | | 30% | 28.7 | 2- |
| Note: Reacts with | | | | | | | | however |
| Ammonia to form | | | | | | | | fire hazard |
| Formamide | | | | | | | | |
| Acetone | 56 | C. | 20.7 | No | | Miscible | 31.3 | 1 |
| Acetonitrile | 81.3 | C. | 37.5 | Yes | | Miscible | 33 | 2 |
| Methanol | 64.7 | C. | 32.6 | No | | Miscible | 38 | 1 |
| Isopropanol | 82.6 | C. | 18.3 | Yes | | Miscible | 44 | 1 |
| Butanone | 79.6 | C. | 18.5 | Yes | | Miscible | 34.7 | 1 |
| Ethanol | 78.37 | C. | 24.3 | Yes | | Miscible | 42.4 | 1 |
| THF | 66 | C. | 7.58 | Yes (95% | | Miscible | 32 | 2 |
| | | | | THF: 5% | | | | |
| | | | | Water) | | | | |
| Acetaldehyde | 20.2 | C. | 21.8 | No | | Miscible | 25.9 | 2, however |
| Reacts with Ammonia | | | | | | | | it is |
| (not in water however) | | | | | | | | carcinogenic |
| | | | | | | | | in humans |
| Propionaldehyde | 46 | C. | 18.9 | No | | 20 g/100 mL | 29.6 | 2 |
| (Propanal) | | | | | | | | |
| Diisopropylamine | 83 | C. | 3.04 | No | 3.43 | Miscible | 34.6 | 2 |
| Dimethoxyethane | 85 | C. | 7.20 | Yes | | Miscible | 36.4 | 2 |
| Dimethoxymethane | 42 | C. | 2.7 | No | | 33% | 29.8 | 2 |
| Tert-Butyl Alcohol | 82 | C. | 10.9 | | | Miscible | | 1 |
| Methyl Acetate | 56.9 | C. | 7.3 | No | | 25% | 32.3 | 1 |
| Note: May react with | | | | | | | | |
| ammonia | | | | | | | | |
| 2-Methyltetrahydrofuran | 80.2 | C. | | | | 14% | | |
| | | | | | | Inversely | | |
| | | | | | | Soluble | | |
| | | | | | | (decreases | | |
| | | | | | | w/increase | | |
| | | | | | | in temp) | | |
| 1 3-Dioxolane | 75 | C. | | Yes (8.7% | | Miscible | | 2 |
| | | | | Water, | | | | |
| | | | | 71.7 C.) | | | | |

Azeotrope Sources, the following are incorporated herein as references:
https://en.wikipedia.org/wiki/Azeotrope_tables
http://vle-calc.com/azeotrope.html
http://chemistry.mdma.ch/hiveboard/picproxie_docs/000506293-azeotropic.pdf
Potential Azeotropes with Methyl Formate+Methanol and Dimethoxymethane:
http://pubs.acs.org/doi/abs/10.1021/je200140m
   More azeotropes containing dimethyloxymethane
   http://www.google.com/patents/U.S. Pat. No. 2,428,815

### Distillation Principles and Processes by Sydney Young

Azeotrope Solvents

| | | | | | | | % of 1st | | Solubility | | | | |
|---|---|---|---|---|---|---|---|---|---|---|---|---|---|
| | | B.P. | | | | Dielectric | by | Mole | in Water | | Enthalpy | Form | |
| | | (2nd | | | | Constant | Weight | Fraction | (2nd | | of Vap. | Azeo-trope | Toxicity |
| 1st | 2nd | Solvent | | B.P. | | (2nd | Azeo- | of 1st | Solvent | | Azeo- | with | (2nd |
| Solvent | Solvent | alone) | | (Azeotrope) | | Solvent) | trope | Solvent | alone) | | trope | Water? | Solvent) |
| Ethanol | Toluene | 110 | C. | 76.7 | C. | 2.38 | 68% | | .52 | g/L | | Yes | 2 |
| | | | | | | | | | | | | (80% T, | |
| | | | | | | | | | | | | 20% H2O) | |
| | Ethyl | 77.1 | C. | 71.8 | C. | 6.02 | 30.8% | | 8.3 g/100 mL | | | Yes (91% | 1 |
| | Acetate | | | | | | | | | | | 2nd) | |
| | Isopropyl | 88.4 | C. | 76.8 | C. | | 53% | | 4.3 g/100 mL | | | | 1 |
| | Acetate | | | | | | | | | | | | |
| Methanol | Dimethoxy | 42 | C. | 41.85 | C. | 2.7 | 8.2% | | 33% | | | | 2 |
| | methane | | | | | | (Weight) | | | | | | |
| | Diethyl | 34.6 | C. | 34.4 | C. | | 3.1% | | | | | | 2 |
| | Ether | | | | | | (Weight) | | | | | | |
| | 2- | 80.2 | C. | 63 | C. | | 49% | | | | | | |
| | Methyltetra | | | | | | (Weight) | | | | | | |
| | hydrofuran | | | | | | | | | | | | |
| | Toluene | 110 | C. | 63.5 | C. | 2.38 | 69% | 0.883 | .52 | g/L | | Yes | 2 |
| | | | | | | | | | | | | (80% T, | |
| | | | | | | | | | | | | 20% H2O) | |
| | Methyl | 56.9 | C. | 53.5 | C. | 7.3 | 19.7% | 0.352 | 25% | | | Yes (95% | 1 |
| | Acetate | | | | | | | | | | | 2nd) | |
| | Ethyl | 77.1 | C. | 62.3 | C. | 6.02 | 44% | | 8.3 g/100 mL | | | | 1 |
| | Acetate | | | | | | | | | | | | |
| | n-Heptane | 98.5 | C. | 59.1 | C. | 1.92 | 51.5% | | Insoluble | | | | |
| | n-Octane | 125.8 | C. | 63 | C. | 1.96 | 28% | | Insoluble | | | | |
| | Acetone | | | 55.7 | C. | | | | | | | | |
| | THF | | | 60.7 | C. | | | | | | | | |
| | Di-n-propyl | | | 62.5 | C. | | | | | | | | |
| | ether | | | | | | | | | | | | |
| | Diisopropyl | | | 54.25 | C. | | | | | | | | |
| | ether | | | | | | | | | | | | |
| | Methyl- | | | 50.1 | C. | | | | 26 | g/L | | | |
| | tert-butyl | | | | | | | | | | | | |
| | ether | | | | | | | | | | | | |
| | Ethyl-n- | | | 54.5 | C. | | | | | | | | |
| | Propyl | | | | | | | | | | | | |
| | Ether | | | | | | | | | | | | |
| Methyl | Methoxy- | 38.8 | C. | 30.5 | C. | | 80% | | 30 | g/L | | | 0 |
| Formate | propane | | | | | | | | | | | | |
| | Methanol | | | | | | | | | | | | |
| Acetone | THF | | | | | | | | | | | | |
| | Diisopropyl | 69 | C. | 53.25 | C. | | | | | | | | 1 |
| | ether | | | | | | | | | | | | |
| | methyl-tert- | | | 49.1 | C. | | | .52 | 26 | g/L | | | |
| | butyl ether | | | | | | | | | | | | |

### Pyrans

### Ternary Azeotropes:

Methanol-Acetone-Methyl Acetate: 53.7 C

Other Added Solvents

| Solvent | Boiling | Dielectric | Azeotrope | | | Enthalpy of | |
|---|---|---|---|---|---|---|---|
| Name | Point | Constant | (Yes/No) | pKa | Solubility | Vap. | Toxicity |
| Glyoxal | 51 C. | | No, however forms | | at least 40% | | 2 |
| | | | complex hydrates | | | | |

Note: amines and other CO₂ reactive compounds may be employed, however, it may be desirable for the amines to not react with the CO₂ absorbent-CO₂, such as in a metathesis reaction.

What is claimed is:

1. An integrated process for capturing CO₂ comprising:
desorbing gaseous CO₂ from a CO₂ containing solution comprising carbonate, bicarbonate, sesquicarbonate, carbamate, or a mixture thereof;
wherein said desorbing of gaseous CO₂ is conducted in the presence of a suitable soluble substance.

2. The integrated process of claim 1 which further comprises at least partially recovering said soluble substance.

3. The integrated process of claim 2 wherein said at least partially recovering comprises employing a membrane capable of at least partially rejecting said soluble substance while allowing substantial passage of CO₂ containing solution.

4. The integrated process of claim 3 wherein said membrane has a molecular weight cutoff of greater than about 80 daltons.

5. The integrated process of claim 3 wherein said membrane is comprised of a material selected from the group consisting of:
a) thin film composite;
b) polyamide;
c) cellulose acetate;
d) ceramic membrane; and
e) combinations thereof.

6. The integrated process of claim 2 wherein said at least partially recovering comprises employing distillation.

7. The integrated process of claim 1 wherein the soluble substance comprises water, organic solvent, water soluble polymer, soluble polymer, glycol, polyethylene glycol, polypropylene glycol, ethers, glycol ethers, glycol ether esters, triglyme, polyethylene glycols of multiple geometries, including, branched polyethylene glycols, star polyethylene glycols, comb polyethylene glycols, methoxypolyethylene glycol, polyvinyl alcohol, polyvinylpyrrolidone, polyacrylic Acid, diol polymers, 1,2 propanediol, 1,2 ethanediol, 1,3 propanediol, cellulose ethers, methylcellulose, cellosize, carboxymethylcellulose, hydroxyethylcellulose, sugar alcohol, sugars, alcohols, ketones, aldehydes, esters, organosilicon compounds, halogenated solvents, non-volatile solvents, a substance with a vapor pressure less than 0.01 atm at 20° C., soluble substances with a molecular weight greater than 80 daltons, or a mixture thereof.

8. The integrated process of claim 1 wherein the soluble substance comprises one or more or a combination of the following: volatile organic solvents, soluble substances with a molecular weight less than 600 daltons, soluble substances with a molecular weight less than 200 daltons, dimethoxymethane, acetone, acetaldehyde, methanol, dimethyl ether, THF, ethanol, isopropanol, propanal, methyl formate, azeotropes, alcohols, ketones, aldehydes, esters, organosilicon compounds, halogenated solvents, a substance with a vapor pressure greater than 0.01 atm at 20° C., or a mixture thereof.

9. The integrated process of claim 7 which further comprises at least partially recovering said soluble substance by employing a membrane capable of at least partially rejecting said soluble substance while allowing substantial passage of CO₂ containing solution.

10. The integrated process of claim 8 which further comprises at least partially recovering said soluble substance by distillation.

11. The integrated process of claim 1 wherein the soluble substance comprises a non-ionic carbon containing compound.

12. The integrated process of claim 1 wherein the soluble substance comprises a thermally switchable substance.

13. The integrated process of claim 1 wherein the soluble substance comprises a CO₂ switchable substance.

14. The integrated process of claim 1 which further comprises capturing CO₂ to form the CO₂ containing solution from a source selected from the group consisting of flue gas; combustion emissions; manufacturing emissions; refining emissions or a combination thereof.

15. The integrated process of claim 1 wherein the CO₂ containing solution further comprises a CO₂ absorbent.

16. The integrated process of claim 15 wherein said absorbent comprises: ammonia, ammonium, amine, amino ethyl ethanol amine, 2-amino-2-methylpropan-1-ol (AMP), MDEA, MEA, primary amine, secondary amine, tertiary amine, low molecular weight primary or secondary amine, metal-amine complex, metal-ammonia complex, metal-ammonium complex, sterically hindered amine, imines, azines, piperazine, alkali metal, lithium, sodium, potassium, rubidium, caesium, alkaline earth metal, calcium, magnesium, ionic liquid, thermally switchable compounds, CO₂ switchable compounds, enzymes, metal-organic frameworks, quaternary ammonium, quaternary ammonium cations, quaternary ammonium cations embedded in polymer, or a mixture thereof.

17. The integrated process of claim 1 wherein CO₂ is desorbed at a temperature of from about 18° C. to about 32° C.

18. The integrated process of claim 1 wherein CO₂ is desorbed at a temperature of less than or equal to about 40° C.

19. The integrated process of claim 1 wherein CO₂ is desorbed at a pressure of from about 0.75 to about 1.25 atmospheres.

20. An integrated process for capturing CO₂ comprising:
capturing CO₂ to with a solution comprising a CO₂ absorbent to form a CO₂ containing solution comprising carbonate, bicarbonate, sesquicarbonate, carbamate, or a mixture thereof;
desorbing gaseous CO₂ from the CO₂ containing solution comprising carbonate, bicarbonate, sesquicarbonate, carbamate, or a mixture thereof wherein said desorbing of gaseous CO₂ is conducted in the presence of a suitable soluble substance; and
at least partially recovering said soluble substance by employing (1) a membrane with a molecular weight cutoff of greater than about 80 daltons or (2) distillation or (3) a combination thereof; wherein said soluble substance comprises water, organic solvent, water soluble polymer, soluble polymer, glycol, polyethylene glycol, polypropylene glycol, ethers, glycol ethers, glycol ether esters, triglyme, polyethylene glycols of multiple geometries, including, branched polyethylene glycols, star polyethylene glycols, comb polyethylene glycols, methoxypolyethylene glycol, polyvinyl alcohol, polyvinylpyrrolidone, polyacrylic Acid, diol polymers, 1,2 propanediol, 1,2 ethanediol, 1,3 propanediol, cellulose ethers, methylcellulose, cellosize, carboxymethylcellulose, hydroxyethylcellulose, sugar alcohol, sugars, alcohols, ketones, aldehydes, esters, organosilicon compounds, halogenated solvents, non-volatile solvents, a substance with a vapor pressure less than 0.01 atm at 20° C., soluble substances with a molecular weight greater than 80 daltons, volatile organic solvents, soluble substances with a molecular weight less than 600 daltons, soluble substances with a molecular weight less than 200 daltons, dimethoxymethane, acetone, acetaldehyde, methanol, dimethyl ether, THF, ethanol, isopropanol, propanal, methyl formate, azeotropes, alcohols, ketones, aldehydes, esters, organosilicon compounds, halogenated solvents, a substance with a vapor pressure greater than than 0.01 atm at 20° C., or a mixture thereof.

21. The integrated process of claim 18 wherein the desorbing of gaseous CO₂ from the CO₂ containing solution occurs in the absence of substantial precipitate formation at a temperature of from about 18° C. to about 32° C. and a pressure of from about 0.75 to about 1.25 atmospheres.

22. The integrated process of claim 18 further comprising producing ammonium carbamate, urea, or a derivative thereof.
